(19) United States
(12) Patent Application Publication
(10) Pub. No.: US 2026/0366044 A1
(43) Pub. Date: Nov. 19, 2026

(54) Method of Enriching for Edited Cells

(71) Applicant: ST. JUDE CHILDREN'S RESEARCH HOSPITAL, INC., Memphis, TN (US)

(72) Inventors: Shondra M. Miller, Memphis, TN (US); Jon P. Connelly, Memphis, TN (US); Jamaica F. Siwak, Memphis, TN (US)

(21) Appl. No.: 19/649,493

(22) Filed: Apr. 16, 2026

Related U.S. Application Data
(60) Provisional application No. 63/804,865, filed on May 13, 2025.

(51) Int. Cl. C12N 15/90; C12N 9/22; C12N 15/11

(52) U.S. Cl. CPC C12N 15/907; C12N 9/226; C12N 15/11; C12N 2310/20

(57) ABSTRACT

A method and system for producing and enriching for edited cells in a population of cells is provided, which is based upon homology directed repair to introduce a silent mutation into an essential gene concurrently with the introduction of an edit at a genomic location of interest such that correct repair of the essential gene functions as a surrogate reporter for editing at the genomic location of interest.

### REFERENCE TO RELATED APPLICATIONS

This application claims the benefit of priority from U.S. Provisional Application Ser. No. 63/804,865, filed May 13, 2025, the content of which is incorporated herein by reference in its entirety.

### BACKGROUND OF THE INVENTION

CRISPR-based genetic engineering is a flexible way of introducing genomic mutations in cells. Double strand DNA (dsDNA) breaks can be induced at desired genomic loci through the use of “programmable”, user-defined short guide RNAs which complex with a nuclease. Frequently used nucleases include Cas proteins, particularly Cas9 or variations thereof. Variations include naturally occurring orthologues such as Cas12a or engineered nucleases with altered DNA binding specificities or fusion proteins that add distinct features such as transcriptional activation or repression or enzymatic activity to directly edit nucleotides. Cas nucleases can also be modified to induce single-stranded “nicks” to genomic DNA. The cellular response to these induced DNA breaks is the activation of the DNA repair machinery, which mainly consists of the non-homologous end joining (NHEJ) pathway and the homology directed repair (HDR) pathway. NHEJ usually results in random insertions and deletions (indels) at the cut site, which can be exploited to delete or knockout genes. This can be useful for experimental purposes, but for clinical use the inherently stochastic NHEJ repair pathway bears significant risks. Targeted, precise gene editing is safer and therefore more desirable. The HDR pathway provides the opportunity to introduce precise mutations by repairing a DNA break based on a DNA template. However, exploiting the HDR pathway for biotechnology purposes is much less efficient than exploiting NHEJ. A bottleneck to overcome the low HDR efficiency is the absence of simple systems to quantitate NHEJ and HDR events in single cells. Many assays to assess gene editing events are semi-quantitative. Sequencing of entire cell populations does not provide information about the frequency of events per cell and does not allow the discrimination of homozygosity vs. heterozygosity. Although cell lines can be cloned to obtain single cell information, this approach is tedious and is not possible in primary cells. Alternatively, flow cytometry-based reporter systems have been developed to quantify gene editing on a single cell basis. However, such systems depend on genetic manipulation of the assessed cells or organisms prior to their use, thus restricting their use.

Accordingly, needed is a simple cost-effective approach, for identifying and enriching for cells comprising a gene editing event under the control of the natural gene elements without the need for transgenes, application of drugs/chemicals, and without the need for prior manipulation of cells. The present methods and compositions address this need in the art.

### SUMMARY OF THE INVENTION

This invention provides a method for producing and enriching for edited cells in a population of cells comprising (a) inducing in the genome of cells of a population a break (i) at a genomic location of interest and (ii) at a genomic location within a coding sequence of a gene encoding an essential protein required for survival and/or proliferation of the cells; (b) providing to the cells an editing construct comprising an exogenous nucleotide sequence of interest flanked by at least two regions of homology to the genomic location of interest; (c) providing to the cells a replacement construct comprising a portion of the coding sequence of the gene encoding the essential protein flanked by at least two regions of homology to the genomic location within the coding sequence of the gene encoding the essential protein, wherein the portion of the coding sequence of the gene encoding the essential protein comprises a silent mutation; and (d) selecting cells of the population of cells that comprise the exogenous nucleotide sequence of interest and express the essential protein thereby producing and enriching for edited cells in the population of cells.

The invention also provides a system for editing the genome of a cell comprising (a) a nuclease that introduces a break at a genomic location of interest and at a genomic location within a coding sequence of a gene encoding an essential protein required for survival and/or proliferation of the cells; (b) an editing construct comprising an exogenous nucleotide sequence of interest flanked by at least two regions of homology to the genomic location of interest; and (c) a replacement construct comprising a portion of the coding sequence of the gene encoding the essential protein flanked by at least two regions of homology to the genomic location within the coding sequence of the gene encoding the essential protein, wherein the portion of the coding sequence of the gene encoding the essential protein comprises a silent mutation.

### BRIEF DESCRIPTION OF DRAWINGS

FIG. 1 shows cell intrinsic selection via double-stranded break repair at essential loci, wherein cells repaired by NHEJ are selected against and cells repaired by HDR, referred to as “HDRescue,” are positively selected for. Shown are the editing outcomes at the target and essential gene locus.

FIG. 2 shows that essential genes do not tolerate frameshift mutations, such that negative selection may occur via NHEJ at essential loci. This analysis was conducted in DLD-1, a colorectal adenocarcinoma cell line.

FIG. 3 shows that selective advantage of using a silent mutation in the coding sequence encoding the essential gene RAN (Ras-related nuclear protein).

FIGS. 4A-4B show targeting with HDRescue, wherein the target gene is AAVS1 (FIG. 4A) and the essential gene is RAN (FIG. 4B).

FIGS. 5A-5B show that splicing factor 3b subunit 1 (SF3B1, FIG. 5A) and glyceraldehyde-3-phosphate dehydrogenase (GAPDH, FIG. 5B) may also be used for HDRescue. This analysis was conducted in U2OS cells.

FIG. 6 shows the enhancement of HDR in induced pluripotent stem cell (iPSC) pools with HDRescue.

FIG. 7 shows that essential HDRescue enriches for iPSC clones with HDR-driven mutations.

FIG. 8 shows that HDRescue can enrich for multi-allelic HDR editing in iPSC clones with HDR-driven mutations.

FIG. 9 shows that HDRescue can enrich for iPSCs with large knock-ins, e.g., a fluorescent protein tag inserted into the C-terminus of the G3BP Stress Granule Assembly Factor 1 (G3BP1) gene.

### DETAILED DESCRIPTION OF THE INVENTION

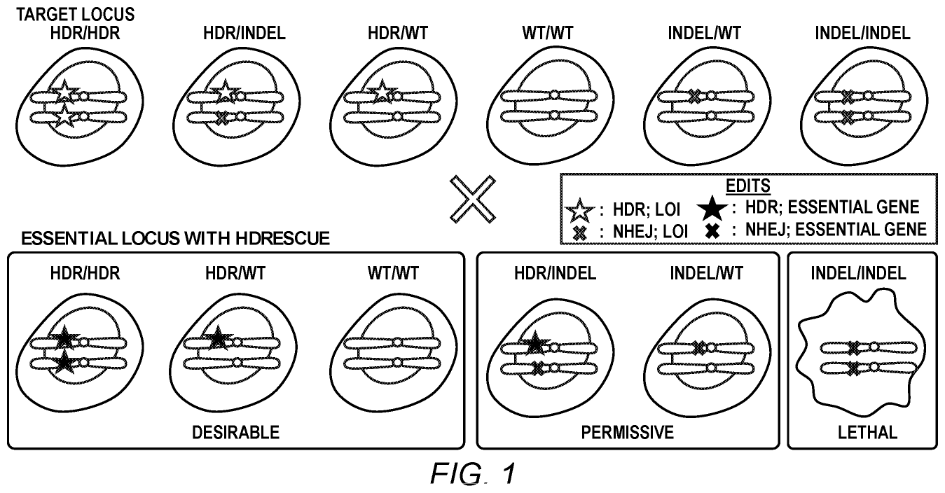

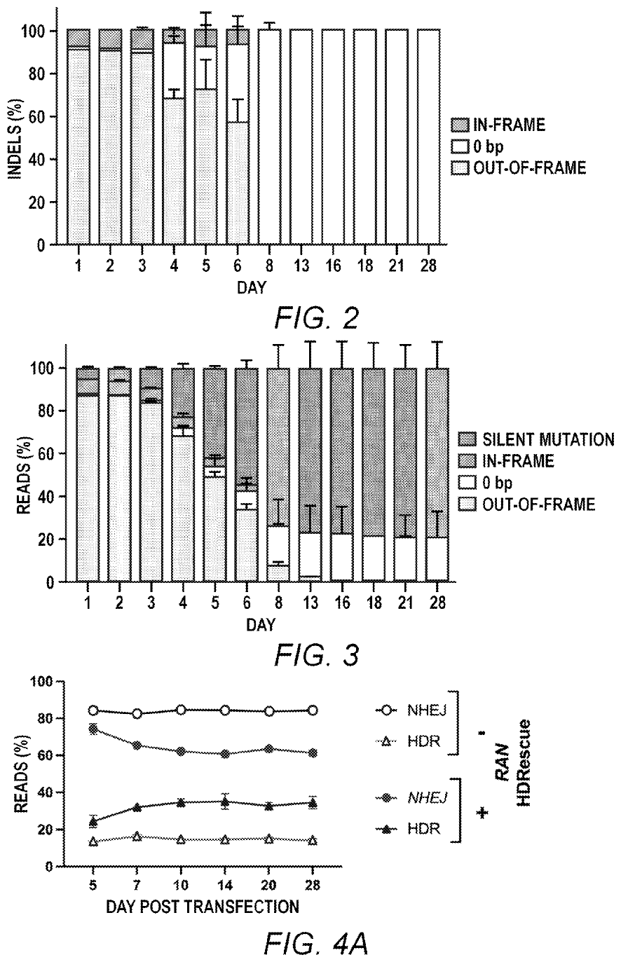

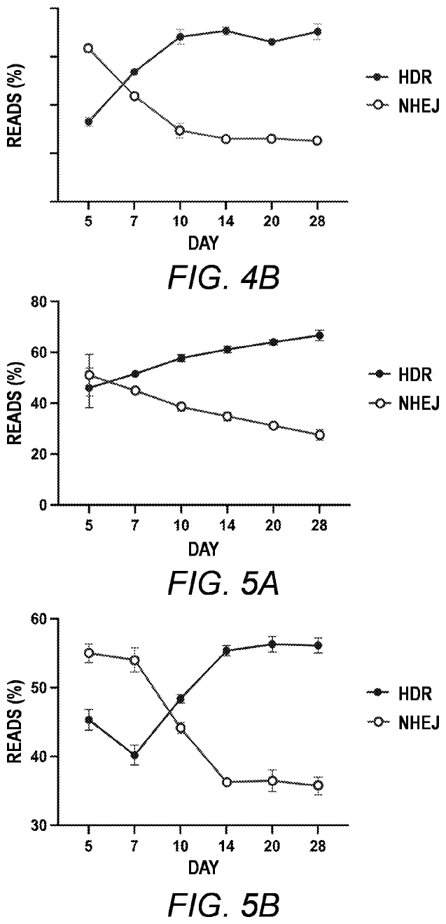

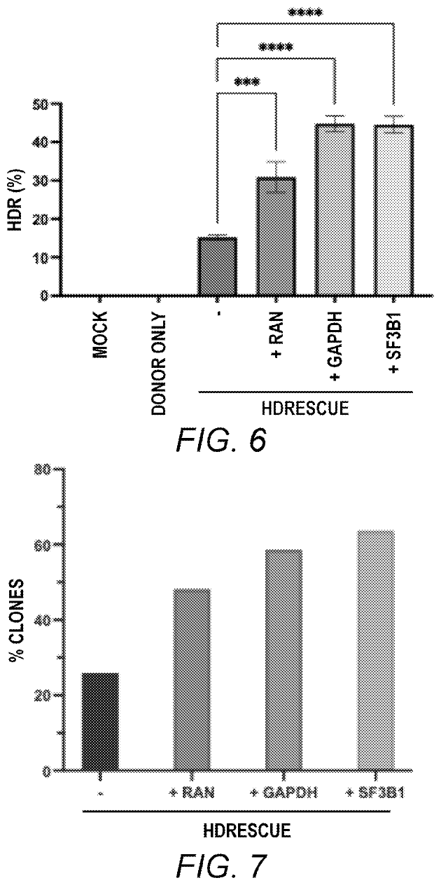

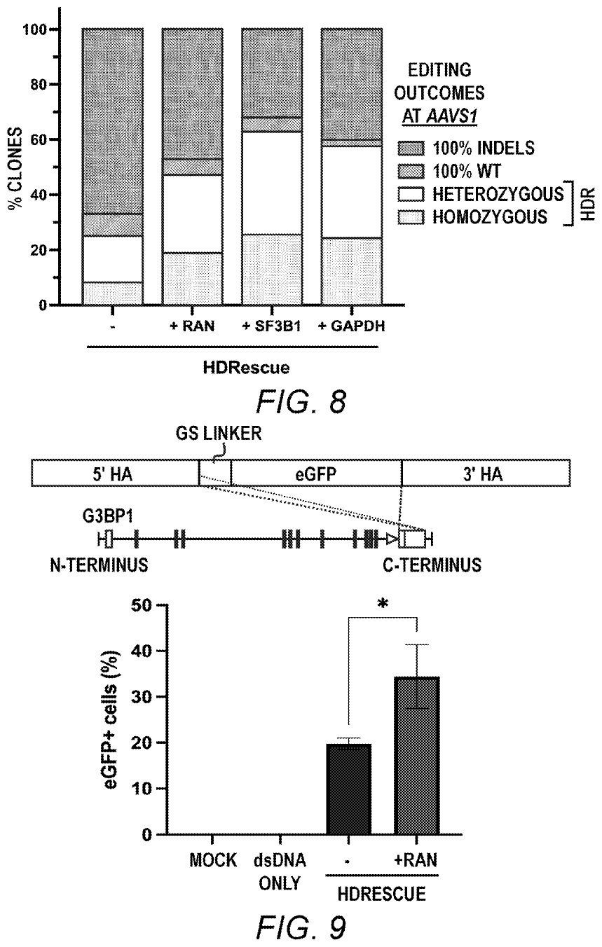

The invention is based, in part, on targeting an essential gene as a surrogate reporter for editing at another location in the genome of a cell. As demonstrated herein, cells were provided with genome editing reagents (e.g., a CRISPR/Cas nuclease, a guide RNA targeting a gene encoding an essential protein, and a guide RNA targeting a genomic location of interest), along with template DNAs for repairing the breaks caused by the nuclease, wherein one template DNA included, e.g., a reporter construct to be introduced into the genomic location of interest and the other template DNA included nucleic acids to repair the break in the gene encoding the essential protein, e.g., an exogenous ssODN that contained a silent mutation. Cells that underwent repair by NHEJ (the majority) died as the essential gene was mutated and cells that repaired the essential gene in a precise fashion via HDR using the provided SSODN survived. See, e.g., FIGS. 1-9. This invention allows for enrichment of cells that have undergone HDR at the essential gene as a surrogate for correct editing at the desired modification site. In comparison, other existing technologies leave undesired genomic changes that have functional outcomes in the cells. Notably, the amino acid sequence of the essential protein will remain the same by introducing at least one silent mutation in the wobble position of the coding sequence of the gene encoding the essential protein. In some aspects, the silent mutation may target naturally occurring single nucleotide polymorphisms (SNPs). Advantageously, the method herein is carried out in a single transformation or transfection (i.e., simultaneously), no drug selection is needed, the edits made at the target site are at the endogenous site and under the control of endogenous regulatory elements, no prior editing of the cells is required to allow the cells to be enriched, and the edit made at the essential gene should have no function consequence as the amino acid sequence of the protein is not altered.

Accordingly, this invention provides a method and system for producing and enriching for edited cells, in particular HDR-mediated edited cells, in a population of cells. The method of the invention comprises the steps of inducing in the genome of cells of a population a break (i) at a genomic location of interest and (ii) at a genomic location within a coding sequence of a gene encoding an essential protein required for survival and/or proliferation of the cells; providing to the cells an editing construct comprising an exogenous nucleotide sequence of interest flanked by at least two regions of homology to the genomic location of interest; providing to the cells a replacement construct comprising a portion of the coding sequence of the gene encoding the essential protein flanked by at least two regions of homology to the genomic location within the coding sequence of the gene encoding the essential protein, wherein the portion of the coding sequence of the gene encoding the essential protein comprises at least one silent mutation; and selecting cells of the population of cells that comprise the exogenous nucleotide sequence of interest and express the essential protein thereby producing and enriching for edited cells in the population of cells.

The term “homology-directed repair” or “HDR” refers to a mechanism in cells to accurately and precisely repair double-strand DNA breaks using a homologous template to guide repair. The most common form of HDR is homologous recombination (HR). The term “homologous recombination” or “HR” refers to a genetic process in which nucleotide sequences are exchanged between two similar molecules of DNA. Homologous recombination (HR) is used by cells to accurately repair harmful breaks that occur on both strands of DNA, known as double-strand breaks or other breaks that generate overhanging sequences.

As used herein, an “edited cell,” “gene modified cell,” or “genome edited cell” refers to a cell into which an exogenous nucleic acid has been introduced into the genomic DNA, in particular at a genomic location of interest. Such an edit in the genome of the cell may be a single nucleotide or base pair substitution, insertion, or deletion; a plurality (e.g., 2, 3, 4, 5, 10, 20, 30 or more) of nucleotide or base pair substitutions, insertions, or deletions; or insertion of an exogenous transgene, when compared to the genome of the cell that does not include the edit.

In some aspects, a “genomic location of interest” may be native or endogenous gene that has a mutation, which leads to a disease phenotype (e.g., sickle cell disease or cystic fibrosis), such that introduction of an editing construct may be used to correct or repair the mutation and treat the disease. In some aspects, a “genomic location of interest” may be a genomic safe harbor site that can accommodate integration of an exogenous nucleotide sequence, e.g., a transgene.

The term “host cell,” “cell,” “population of cells,” or grammatical variations thereof, refers to a cell or cells targeted for genetic modification or editing. In some aspects, the genome of the cell or population of cells does not comprise a non-natural (e.g., man-made or artificial) mutation or transgene, e.g., a reporter gene or an antibiotic resistance gene.

In some aspects, a cell is a tumor or artificially immortalized cell line. In some aspects, a cell used herein may be a primary cell or population of primary cells. The term “primary cell” refers to a cell isolated directly from a multicellular organism. Primary cells typically have undergone very few population doublings and are therefore more representative of the main functional component of the tissue from which they are derived in comparison to continuous (tumor or artificially immortalized) cell lines.

In some aspects, a cell herein is a progenitor or stem cell, e.g., a primary progenitor or embryonic stem (ES) cell. In some aspects, the progenitor or stem cell is a hematopoietic stem cell, a hematopoietic progenitor cell, a myeloid progenitor cell, a lymphoid progenitor cell, a multipotent progenitor, an oligopotent progenitor, or a lineage-restricted progenitor. In some aspects, a cell herein is a blood cell, e.g., a erythrocyte (e.g., red blood cell) or a type of cell that can become an erythrocyte such as a mature erythrocyte that does not contain a nucleus. Non-limiting examples of cells that can become erythrocytes include hemocytoblasts (e.g., blood stem cells or hematopoietic stem cells), hematopoietic progenitor cells, common myeloid progenitor cells, proerythroblasts, basophilic erythroblasts, intermediate erythroblasts, late erythroblasts, reticulocyte, and the like. In some aspects, a cell herein is a mesenchymal cell, e.g., a primary mesenchymal cell including primary cells of a mesenchymal lineage that can differentiate to become cells of bone, cartilage, fat, muscle, etc. Non-limiting examples include mesenchymal stem cells (i.e., mesenchymal stromal cells or MSCs), mesenchymal progenitor cells, and mesenchymal precursor cells, as well as differentiated mesenchymal cells such as bone cells, cartilage cells, muscle cells, adipose cells, stromal cells, fibroblasts, and/or dermal cells.

Primary cells may be harvested from a subject by any standard method. For instance, cells from tissues, such as skin, muscle, bone marrow, spleen, liver, kidney, pancreas, lung, intestine, stomach, etc., may be harvested by a tissue biopsy or a fine needle aspirate. Blood cells and/or immune cells may be isolated from whole blood, plasma, or serum. Induced pluripotent stem (iPS) cells may be generated from differentiated cells according to standard protocols described in, e.g., U.S. Pat. Nos. 8,871,504, 8,900,871 and 8,791,248. In some aspects, the primary cell is in vitro. In some aspects, the primary cell is ex vivo.

As used herein, an “exogenous” or “heterologous” molecule, construct, or sequence refers to a molecule, construct, or sequence, or portion of a nucleic acid molecule, that is not native to a host cell, but may be homologous to a molecule, construct, or sequence, or portion of a nucleic acid molecule from the host cell. The source of the heterologous or exogenous molecule, construct, or sequence may be from a different genus or species. In some aspects, a exogenous or heterologous molecule, construct, or sequence is introduced or provided (i.e., not endogenous or native) to a host cell or host genome by, for example, conjugation, transformation, transfection, electroporation, for the like, wherein the exogenous or heterologous molecule, construct, or sequence, or portion thereof, may integrate into the host genome.

In some aspects, an “exogenous nucleotide sequence of interest” is a sequence that is used to introduce an edit or modification into a genomic location of interest. In some aspects, an “exogenous nucleotide sequence of interest” may be an oligonucleotide, e.g., a single-stranded oligodeoxynucleotide (SSODN), having a size in the range of about 40 to about 500 nucleotides (e.g., about 40, 50, 60, 70, 80, 90, 100, 120, 140, 160, 180, 200, 220, 240, 260, 280, 300, 320, 340, 360, 380, 400, 420, 440, 460, 480, or 500 nucleotides, or any range therebetween). In some aspects, an exogenous single-stranded oligodeoxynucleotide may be used to introduce a single or plurality of nucleotide substitutions, insertions, or deletions in a genomic location of interest, e.g., to repair or mutate a genomic location of interest.

In some aspects, an “exogenous nucleotide sequence of interest” is a transgene. In some aspects, a transgene may include a coding sequence encoding a protein or RNA of interest and/or any number of transcription and translation control elements, including promoter, transcription enhancers, transcription terminators, and the like. In some aspects, a transgene may encode an mRNA, siRNA, dsRNA, miRNA, shRNA, a native polypeptide (i.e., a polypeptide found in nature) or fragment thereof; a variant polypeptide (i.e., a mutant of the native polypeptide having less than 100% sequence identity with the native polypeptide) or fragment thereof, an engineered polypeptide or peptide fragment, a therapeutic peptide or polypeptide, an imaging marker, a selectable marker, a degradation signal, and the like. For example, a transgene may encode an antibody, an antigen, an enzyme, a growth factor, a receptor (e.g., cell surface, cytoplasmic, or nuclear), a hormone, a lymphokine, a cytokine, a chemokine, a reporter, a functional fragment of any of the above, or a combination of any of the above. A transgene may also include a chimeric antigen receptor (CAR). The term “chimeric antigen receptor” refers to an engineered receptor comprising domains of T cell receptors and B cell receptors. CARs recognize a wide range of antigens comprising proteins, lipids and sugars. In instances where the transgene encodes a CAR, the ligand is the respective antigen.

To achieve efficient integration of an exogenous nucleotide sequence of interest into a genomic location of interest by HDR, the exogenous nucleotide sequence of interest may be provided or introduced into a cell as a component of an editing construct (e.g., a template DNA) comprising the exogenous nucleotide sequence of interest flanked by at least two regions of homology (homology arms) that are homologous to a genomic location of interest. In some aspects, the at least two regions of homology to the genomic location of interest correspond to sequences located on either side (5′ and 3′) of a break at the genomic location of interest.

To facilitate the identification of cells that comprise the exogenous nucleotide sequence of interest at a genomic location of interest, the method further provides for editing an essential gene as a surrogate reporter for editing at genomic location of interest. Accordingly, in addition to an editing construct, the cells are also provided a replacement construct (e.g., a donor DNA or template DNA) comprising a portion of the coding sequence of a gene encoding an essential protein flanked by at least two regions of homology (homology arms) to the genomic location within the coding sequence of an gene encoding an essential protein, wherein the portion of the coding sequence of the gene encoding the essential protein comprises at least one silent mutation. In some aspects, the at least two regions of homology to the genomic location within the coding sequence of the gene encoding the essential protein correspond to sequences located on either side (5′ and 3′) of a break at the genomic location within a coding sequence of the gene encoding the essential protein.

In some aspects, a “region of homology” refers to a nucleotide sequence capable of hybridizing with a complementary nucleotide sequence at a genomic location (e.g., a genomic location of interest or a genomic location within a coding sequence of a gene encoding an essential protein). The region of homology may be a full complement or less than fully complementary to a nucleotide sequence at the genomic location as long as it is capable of hybridizing to the nucleotide sequence at the genomic location under physiological conditions. A “full complement” or “full-length complement” refers to a sequence that shares 100% sequence identity with another sequence. A sequence that is less than fully complementary may differ by one or more nucleotides. In some aspects, a region of homology shares at least about 80% sequence identity with a nucleotide sequence at a genomic location (e.g., at least about 85%, 90%, 95%, 97%, 98%, 99%, or 100% identity).

The terms “sequence identity” or “percent identity” in the context of two or more nucleic acids or polypeptides refer to two or more sequences or subsequences that are the same (“identical”) or have a specified percentage of amino acid residues or nucleotides that are identical (“percent identity”) when compared and aligned for maximum correspondence with a second molecule, as measured using a sequence comparison algorithm (e.g., by a BLAST alignment, or any other algorithm), or alternatively, by visual inspection.

As used herein, a nucleotide sequence “flanked by at least two regions of homology,” means that the at least two regions of homology are positioned on either side of the reference nucleotide sequence, wherein at least one region of homology is located on the 5′ end of reference nucleotide sequence and at least one region is located on the 3′ end of reference nucleotide sequence. In some aspects, a construct herein has the structure: 5′ homology arm-reference nucleotide sequence-3′ homology arm. In some aspects, regions of homology that flank a reference nucleotide sequence (e.g., exogenous nucleotide sequence of interest and/or a portion of a coding sequence of a gene encoding an essential protein) may comprise at least 85 nucleotides each. In some aspects, regions of homology herein comprise at least 450 nucleotides each. In some aspects, regions of homology herein comprise approximately 2000 nucleotides each. In some aspects, regions of homology herein are at least 2000 nucleotides each, e.g., about 3000 nucleotides in length. In some aspects, regions of homology herein are independently at least about 90, 100, 120, 140, 160, 180, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, or 3000 nucleotides each, or any range therebetween. In some aspects, the at least two regions of homology may have the same length, or may differ in length. The selection of appropriate homology arm lengths may be influenced by a variety of factors, such as the desire to avoid homologies or microhomologies with certain sequences such as Alu repeats or other very common elements. In some aspects, the regions of homology (e.g., 5′ and 3′ homology arms) flank the break and are less than 100, 75, 50, 25, 15, 10 or 5 nucleotides away from an edge of the break. In some aspects, one or more of the regions of homology (e.g., 5′ and/or 3′ homology arm) encompasses an edge of the break.

As used herein, an “essential protein” is a protein required for survival and/or proliferation of the cells used herein. An essential protein may be a protein encoded by a housekeeping gene that is essential for survival of all cell types or a protein that is required to be expressed in a specific cell type for survival and/or proliferation under particular culture conditions, e.g., for proper differentiation of iPS or ES cells or expansion of iPS- or ES-derived cells. In some aspects, loss of function of an essential gene encoding an essential protein results in a significant reduction of cell survival, e.g., of the time a cell characterized by a loss of function of an essential gene survives as compared to a cell of the same cell type but without a loss of function of the same essential gene. In some aspects, loss of function of an essential gene results in the death of the affected cell. In some aspects, loss of function of an essential gene results in a significant reduction of cell proliferation, e.g., in the ability of a cell to divide, which may manifest in a significant time period the cell requires to complete a cell cycle, or, in some aspects, in a loss of a cell's ability to complete a cell cycle, and thus to proliferate at all. Accordingly, in some aspects, a break (e.g., a double-strand break) is induced or introduced into a genomic location within the coding sequence of an essential gene. In accordance with this aspect, if a portion of the coding sequence of the gene encoding the essential protein is not integrated into the genome of the cells by HDR in the correct position or orientation, the cells do not survive and/or proliferate.

In some aspects, a “coding sequence of a gene encoding an essential protein” includes exonic and intronic sequences. In some aspects, a “coding sequence of a gene encoding an essential protein” includes only exonic sequences. In some aspects, a “coding sequence of a gene encoding an essential protein” refers to gene sequence occurring at or 3′ of the endogenous translation start coding (e.g., ATG) and at or 5′ of an endogenous translation stop signal (e.g., TAA, TAG or TGA).

In order to properly restore the essential gene coding sequence in the edited cell (so that a functioning gene product is produced), the replacement construct (donor DNA or template DNA) does not need to comprise an exogenous coding sequence that corresponds to the entire coding sequence of the essential gene. Depending on the location of the break in the endogenous coding sequence of the essential gene it may be possible to restore the essential gene by providing a replacement construct that comprises a portion of the coding sequence of the essential gene, e.g., that corresponds to a portion of the endogenous coding sequence of the essential gene that spans a break. Accordingly, a “portion of the coding sequence of the gene encoding the essential protein” may be as few as one nucleotide or may include essentially the entire coding sequence. In some aspects, “portion of the coding sequence of the gene encoding the essential protein” is in the range of 1 and 100 nucleotides in length, e.g., 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, or 100 nucleotides in length, or any range therebetween.

In some aspects, the portion of the coding sequence of the gene encoding the essential protein comprises at least one silent mutation, e.g., 1, 2, 3, 4 or more silent mutations. In some aspects, a “silent mutation” refers to a change (e.g., a point mutation) in a coding sequence that does not alter the sequence and/or function of the protein encoded thereby. For example, the codon CUU codes for the amino acid leucine. Replacing or mutating the third position (wobble position) of the CTT codon to provide a CTC, CTA, or CTG codon will also code for leucine. In some aspects, silent mutations (also referred to as synonymous mutations) may be described as single-nucleotide polymorphisms (SNPs) that result in no change to the amino acid sequence because of codon redundancy or degeneracy in the genetic code. In some aspects, a silent mutation may occur in a non-coding region (e.g., within an intron). Exemplary silent mutations may be introduced by replacing a native or wild-type codon with an alternative codon as provided in Table 1.

TABLE 1

| | | Second base in codon | | | | | | | | | |
|---|---|---|---|---|---|---|---|---|---|---|---|
| | | U | | C | | A | | G | | | |
| First | U | UUU | Phe | UCU | Ser | UAU | Tyr | UGU | Cys | U | Last |
| base | | UUC | | UCC | | UAC | | UGC | | C | base |
| in | | UUA | Leu | UCA | | UAA | Stop | UGA | Stop | A | in |
| codon | | UUG | | UCG | | UAG | | UGG | Trp | G | codon |
| | C | CUU | Leu | CCU | Pro | CAU | His | CGU | Arg | U | |
| | | CUC | | CCC | | CAC | | CGC | | C | |
| | | CUA | | CCA | | CAA | Gln | CGA | | A | |
| | | CUG | | CCG | | CAG | | CGG | | G | |
| | A | AUU | Ile | ACU | Thr | AAU | Asn | AGU | Ser | U | |
| | | AUC | | ACC | | AAC | | AGC | | C | |
| | | AUA | | ACA | | AAA | Lys | AGA | Arg | A | |
| | | AUG | Met | ACG | | AAG | | AGG | | G | |
| | G | GUU | Val | GCU | Ala | GAU | Asp | GGU | Gly | U | |
| | | GUC | | GCC | | GAC | | GGC | | C | |
| | | GUA | | GCA | | GAA | Glu | GGA | | A | |
| | | GUG | | GCG | | GAG | | GGG | | G | |

A gene encoding an essential protein, also referred to herein as an essential gene, may be any gene that is essential for the survival and/or the proliferation of the cell. In some aspects, an essential gene is a housekeeping gene, e.g., a gene listed in Table 2. See also other housekeeping genes discussed in Eisenberg (2014) Trends in Genetics 30(3):119-20.

TABLE 2

| | Gene | | |
|---|---|---|---|
| Ensembl ID | Symbol | Ensembl ID | Gene Symbol |
| ENSG00000075624 | ACTB | ENSG00000231500 | RPS18 |
| ENSG00000116459 | ATP5F1 | ENSG00000112592 | TBP |
| ENSG00000166710 | B2M | ENSG00000072274 | TFRC |
| ENSG00000111640 | GAPDH | ENSG00000164924 | YWHAZ |
| ENSG00000169919 | GUSB | ENSG00000089157 | RPLP0 |
| ENSG00000165704 | HPRT1 | ENSG00000142541 | RPL13A |
| ENSG00000102144 | PGK1 | ENSG00000147604 | RPL7 |
| ENSG00000196262 | PPIA | ENSG00000205250 | E2F4 |
| ENSG00000138160 | KIF11 | ENSG00000160211 | G6PD |

Additional genes that are essential for various cell types, including iPSCs/ESCs, are listed in Table 3 (see also the essential genes discussed in Yilmaz et al. (2018) Nat. Cell Biol. 20:610-619).

TABLE 3

| Ensembl ID | Gene Symbol | Ensembl ID | Gene Symbol |
|---|---|---|---|
| ENSG00000111704 | NANOG | ENSG00000181449 | SOX2 |
| ENSG00000179059 | ZFP42 | ENSG00000136997 | MYC |
| ENSG00000136826 | KLF4 | ENSG00000175166 | PSMD2 |
| ENSG00000118655 | DCLRE1B | ENSG00000070614 | NDST1 |
| ENSG00000172409 | CLP1 | ENSG00000115484 | CCT4 |
| ENSG00000082898 | XPO1 | ENSG00000100890 | KIAA0391 |
| ENSG00000114867 | EIF4G1 | ENSG00000149474 | CSRP2BP |
| ENSG00000115866 | DARS | ENSG00000102738 | MRPS31 |
| ENSG00000204628 | GNB2L1 | ENSG00000136104 | RNASEH2B |
| ENSG00000198242 | RPL23A | ENSG00000106246 | PTCD1 |
| ENSG00000158526 | TSR2 | ENSG00000248919 | ATP5J2-PTCD1 |
| ENSG00000125450 | NUP85 | ENSG00000138663 | COPS4 |
| ENSG00000134371 | CDC73 | ENSG00000115368 | WDR75 |
| ENSG00000164941 | INTS8 | ENSG00000128564 | VGF |
| ENSG00000055483 | USP36 | ENSG00000128191 | DGCR8 |
| ENSG00000258366 | RTEL1 | ENSG00000008294 | SPAG9 |
| ENSG00000188846 | RPL14 | ENSG00000131475 | VPS25 |
| ENSG00000247626 | MARS2 | ENSG00000105523 | FAM83E |
| ENSG00000095787 | WAC | ENSG00000172269 | DPAGT1 |
| ENSG00000108094 | CUL2 | ENSG00000170312 | CDK1 |
| ENSG00000185946 | RNPC3 | ENSG00000104131 | EIF3J |
| ENSG00000154473 | BUB3 | ENSG00000150753 | CCT5 |
| ENSG00000204394 | VARS | ENSG00000140443 | IGF1R |
| ENSG00000103051 | COG4 | ENSG00000010292 | NCAPD2 |
| ENSG00000104738 | MCM4 | ENSG00000171763 | SPATA5L1 |
| ENSG00000117222 | RBBP5 | ENSG00000180098 | TRNAU1AP |
| ENSG00000082516 | GEMIN5 | ENSG00000168374 | ARF4 |
| ENSG00000100162 | CENPM | ENSG00000173812 | EIF1 |
| ENSG00000141456 | PELP1 | ENSG00000100554 | ATP6V1D |
| ENSG00000137807 | KIF23 | ENSG00000072756 | TRNT1 |
| ENSG00000112685 | EXOC2 | ENSG00000135372 | NAT10 |
| ENSG00000125995 | ROMO1 | ENSG00000178394 | HTR1A |
| ENSG00000136891 | TEX10 | ENSG00000128272 | ATF4 |
| ENSG00000173113 | TRMT112 | ENSG00000204070 | SYS1 |
| ENSG00000075914 | EXOSC7 | ENSG00000137815 | RTF1 |
| ENSG00000119523 | ALG2 | ENSG00000198026 | ZNF335 |
| ENSG00000244038 | DDOST | ENSG00000117410 | ATP6V0B |
| ENSG00000108175 | ZMIZ1 | ENSG00000112739 | PRPF4B |
| ENSG00000129691 | ASH2L | ENSG00000129347 | KRI1 |
| ENSG00000183207 | RUVBL2 | ENSG00000221818 | EBF2 |
| ENSG00000055044 | NOP58 | ENSG00000198431 | TXNRD1 |
| ENSG00000204315 | FKBPL | ENSG00000104979 | C19orf53 |
| ENSG00000187522 | HSPA14 | ENSG00000136709 | WDR33 |
| ENSG00000169375 | SIN3A | ENSG00000149100 | EIF3M |
| ENSG00000143748 | NVL | ENSG00000125835 | SNRPB |
| ENSG00000021776 | AQR | ENSG00000116698 | SMG7 |
| ENSG00000132467 | UTP3 | ENSG00000087586 | AURKA |
| ENSG00000087470 | DNM1L | ENSG00000169230 | PRELID1 |
| ENSG00000130811 | EIF3G | ENSG00000143799 | PARP1 |
| ENSG00000180198 | RCC1 | ENSG00000146731 | CCT6A |
| ENSG00000101407 | TTI1 | ENSG00000163877 | SNIP1 |
| ENSG00000116455 | WDR77 | ENSG00000215421 | ZNF407 |
| ENSG00000135763 | URB2 | ENSG00000197724 | PHF2 |
| ENSG00000133316 | WDR74 | ENSG00000172590 | MRPL52 |
| ENSG00000189091 | SF3B3 | ENSG00000175203 | DCTN2 |
| ENSG00000109917 | ZNF259 | ENSG00000149273 | RPS3 |
| ENSG00000130640 | TUBGCP2 | ENSG00000204822 | MRPL53 |
| ENSG00000011376 | LARS2 | ENSG00000109775 | UFSP2 |
| ENSG00000135249 | RINT1 | ENSG00000165733 | BMS1 |
| ENSG00000126883 | NUP214 | ENSG00000104671 | DCTN6 |
| ENSG00000163510 | CWC22 | ENSG00000175224 | ATG13 |
| ENSG00000101138 | CSTF1 | ENSG00000142541 | RPL13A |
| ENSG00000104221 | BRF2 | ENSG00000173805 | HAP1 |
| ENSG00000125630 | POLR1B | ENSG00000115750 | TAF1B |
| ENSG00000083896 | YTHDC1 | ENSG00000165688 | PMPCA |
| ENSG00000105726 | ATP13A1 | ENSG00000159720 | ATP6V0D1 |
| ENSG00000105618 | PRPF31 | ENSG00000074201 | CLNS1A |
| ENSG00000117748 | RPA2 | ENSG00000158417 | EIF5B |
| ENSG00000143294 | PRCC | ENSG00000196588 | MKL1 |
| ENSG00000156239 | N6AMT1 | ENSG00000138614 | VWA9 |
| ENSG00000143384 | MCL1 | ENSG00000124571 | XPO5 |
| ENSG00000113407 | TARS | ENSG00000198000 | NOL8 |
| ENSG00000086589 | RBM22 | ENSG00000181991 | MRPS11 |
| ENSG00000133119 | RFC3 | ENSG00000149823 | VPS51 |
| ENSG00000052749 | RRP12 | ENSG00000151348 | EXT2 |
| ENSG00000103047 | TANGO6 | ENSG00000162396 | PARS2 |
| ENSG00000142751 | GPN2 | ENSG00000204843 | DCTN1 |
| ENSG00000101057 | MYBL2 | ENSG00000177302 | TOP3A |
| ENSG00000176915 | ANKLE2 | ENSG00000142684 | ZNF593 |
| ENSG00000071127 | WDR1 | ENSG00000074800 | ENO1 |
| ENSG00000106344 | RBM28 | ENSG00000167513 | CDT1 |
| ENSG00000100316 | RPL3 | ENSG00000141101 | NOB1 |
| ENSG00000139131 | YARS2 | ENSG00000047315 | POLR2B |
| ENSG00000182831 | C16orf72 | ENSG00000131966 | ACTR10 |
| ENSG00000167325 | RRM1 | ENSG00000115875 | SRSF7 |
| ENSG00000172262 | ZNF131 | ENSG00000186141 | POLR3C |
| ENSG00000007168 | PAFAH1B1 | ENSG00000108424 | KPNB1 |
| ENSG00000117174 | ZNHIT6 | ENSG00000111845 | PAK1IP1 |
| ENSG00000196497 | IPO4 | ENSG00000148832 | PAOX |
| ENSG00000188566 | NDOR1 | ENSG00000156017 | C9orf41 |
| ENSG00000183091 | NEB | ENSG00000198901 | PRC1 |
| ENSG00000011304 | PTBP1 | ENSG00000134001 | EIF2S1 |
| ENSG00000109805 | NCAPG | ENSG00000146918 | NCAPG2 |
| ENSG00000123154 | WDR83 | ENSG00000144713 | RPL32 |
| ENSG00000147416 | ATP6V1B2 | ENSG00000185122 | HSF1 |
| ENSG00000163961 | RNF168 | ENSG00000167658 | EEF2 |
| ENSG00000163811 | WDR43 | ENSG00000164190 | NIPBL |
| ENSG00000143624 | INTS3 | ENSG00000163902 | RPN1 |
| ENSG00000101161 | PRPF6 | ENSG00000244045 | TMEM199 |
| ENSG00000130726 | TRIM28 | ENSG00000143476 | DTL |
| ENSG00000165494 | PCF11 | ENSG00000149503 | INCENP |
| ENSG00000053900 | ANAPC4 | ENSG00000071243 | ING3 |
| ENSG00000168255 | POLR2J3 | ENSG00000186073 | C15orf41 |
| ENSG00000129534 | MIS18BP1 | ENSG00000088836 | SLC4A11 |
| ENSG00000164754 | RAD21 | ENSG00000136273 | HUS1 |
| ENSG00000120158 | RCL1 | ENSG00000005007 | UPF1 |
| ENSG00000161016 | RPL8 | ENSG00000070010 | UFD1L |
| ENSG00000030066 | NUP160 | ENSG00000106263 | EIF3B |
| ENSG00000099624 | ATP5D | ENSG00000213024 | NUP62 |
| ENSG00000116120 | FARSB | ENSG00000067191 | CACNB1 |
| ENSG00000115233 | PSMD14 | ENSG00000179091 | CYC1 |
| ENSG00000086504 | MRPL28 | ENSG00000113312 | TTC1 |
| ENSG00000160752 | FDPS | ENSG00000085831 | TTC39A |
| ENSG00000049541 | RFC2 | ENSG00000118197 | DDX59 |
| ENSG00000148688 | RPP30 | ENSG00000134871 | COL4A2 |
| ENSG00000114573 | ATP6V1A | ENSG00000088986 | DYNLL1 |
| ENSG00000086200 | IPO11 | ENSG00000138778 | CENPE |
| ENSG00000119720 | NRDE2 | ENSG00000106244 | PDAP1 |
| ENSG00000058262 | SEC61A1 | ENSG00000177600 | RPLP2 |
| ENSG00000073111 | MCM2 | ENSG00000112081 | SRSF3 |
| ENSG00000138160 | KIF11 | ENSG00000100413 | POLR3H |
| ENSG00000215193 | PEX26 | ENSG00000172508 | CARNS1 |
| ENSG00000161057 | PSMC2 | ENSG00000147123 | NDUFB11 |
| ENSG00000187514 | PTMA | ENSG00000119953 | SMNDC1 |
| ENSG00000135829 | DHX9 | ENSG00000111640 | GAPDH |
| ENSG00000058729 | RIOK2 | ENSG00000117899 | MESDC2 |
| ENSG00000110330 | BIRC2 | ENSG00000075624 | ACTB |
| ENSG00000141759 | TXNL4A | ENSG00000163166 | IWS1 |
| ENSG00000166986 | MARS | ENSG00000114503 | NCBP2 |
| ENSG00000153774 | CFDP1 | ENSG00000198522 | GPN1 |
| ENSG00000130177 | CDC16 | ENSG00000099899 | TRMT2A |
| ENSG00000241553 | ARPC4 | ENSG00000181544 | FANCB |
| ENSG00000132604 | TERF2 | ENSG00000136982 | DSCC1 |
| ENSG00000114982 | KANSL3 | ENSG00000068366 | ACSL4 |
| ENSG00000213780 | GTF2H4 | ENSG00000062716 | VMP1 |
| ENSG00000139343 | SNRPF | ENSG00000111802 | TDP2 |
| ENSG00000101189 | MRGBP | ENSG00000185627 | PSMD13 |
| ENSG00000079246 | XRCC5 | ENSG00000020426 | MNAT1 |
| ENSG00000196943 | NOP9 | ENSG00000113734 | BNIP1 |
| ENSG00000122965 | RBM19 | ENSG00000102241 | HTATSF1 |
| ENSG00000132383 | RPA1 | ENSG00000160789 | LMNA |
| ENSG00000094880 | CDC23 | ENSG00000062822 | POLD1 |
| ENSG00000213639 | PPP1CB | ENSG00000168944 | CEP120 |
| ENSG00000109911 | ELP4 | ENSG00000139718 | SETD1B |
| ENSG00000180957 | PITPNB | ENSG00000132792 | CTNNBL1 |
| ENSG00000122257 | RBBP6 | ENSG00000173540 | GMPPB |
| ENSG00000173145 | NOC3L | ENSG00000128789 | PSMG2 |
| ENSG00000179115 | FARSA | ENSG00000196365 | LONP1 |
| ENSG00000105171 | POP4 | ENSG00000160214 | RRP1 |
| ENSG00000148303 | RPL7A | ENSG00000179041 | RRS1 |
| ENSG00000167508 | MVD | ENSG00000143106 | PSMA5 |
| ENSG00000115541 | HSPE1 | ENSG00000168411 | RFWD3 |
| ENSG00000170445 | HARS | ENSG00000073584 | SMARCE1 |
| ENSG00000168496 | FEN1 | ENSG00000175334 | BANF1 |
| ENSG00000141367 | CLTC | ENSG00000077152 | UBE2T |
| ENSG00000087191 | PSMC5 | ENSG00000173611 | SCAI |
| ENSG00000163159 | VPS72 | ENSG00000171720 | HDAC3 |
| ENSG00000130741 | EIF2S3 | ENSG00000182197 | EXT1 |
| ENSG00000168495 | POLR3D | ENSG00000114346 | ECT2 |
| ENSG00000071894 | CPSF1 | ENSG00000124214 | STAU1 |
| ENSG00000058600 | POLR3E | ENSG00000126254 | RBM42 |
| ENSG00000100726 | TELO2 | ENSG00000127184 | COX7C |
| ENSG00000165501 | LRR1 | ENSG00000174276 | ZNHIT2 |
| ENSG00000113575 | PPP2CA | ENSG00000177971 | IMP3 |
| ENSG00000116922 | C1orf109 | ENSG00000104872 | PIH1D1 |
| ENSG00000073712 | FERMT2 | ENSG00000132155 | RAF1 |
| ENSG00000174437 | ATP2A2 | ENSG00000163872 | YEATS2 |
| ENSG00000176407 | KCMF1 | ENSG00000119906 | FAM178A |
| ENSG00000140525 | FANCI | ENSG00000217930 | PAM16 |
| ENSG00000101182 | PSMA7 | ENSG00000197498 | RPF2 |
| ENSG00000130204 | TOMM40 | ENSG00000130348 | QRSL1 |
| ENSG00000239306 | RBM14 | ENSG00000147536 | GINS4 |
| ENSG00000248643 | RBM14-RBM4 | ENSG00000174748 | RPL15 |
| ENSG00000172113 | NME6 | ENSG00000159147 | DONSON |
| ENSG00000136448 | NMT1 | ENSG00000157593 | SLC35B2 |
| ENSG00000186166 | CCDC84 | ENSG00000181938 | GINS3 |
| ENSG00000166233 | ARIH1 | ENSG00000187446 | CHP1 |
| ENSG00000111877 | MCM9 | ENSG00000070371 | CLTCL1 |
| ENSG00000204316 | MRPL38 | ENSG00000096063 | SRPK1 |
| ENSG00000101868 | POLA1 | ENSG00000141564 | RPTOR |
| ENSG00000107951 | MTPAP | ENSG00000108474 | PIGL |
| ENSG00000039650 | PNKP | ENSG00000187741 | FANCA |
| ENSG00000123064 | DDX54 | ENSG00000213465 | ARL2 |
| ENSG00000183955 | SETD8 | ENSG00000117593 | DARS2 |
| ENSG00000138107 | ACTR1A | ENSG00000171863 | RPS7 |
| ENSG00000244005 | NFS1 | ENSG00000117395 | EBNA1BP2 |
| ENSG00000188986 | NELFB | ENSG00000111142 | METAP2 |
| ENSG00000018699 | TTC27 | ENSG00000113272 | THG1L |
| ENSG00000167112 | TRUB2 | ENSG00000117360 | PRPF3 |
| ENSG00000100393 | EP300 | ENSG00000221978 | CCNL2 |
| ENSG00000101639 | CEP192 | ENSG00000163832 | ELP6 |
| ENSG00000126461 | SCAF1 | ENSG00000108852 | MPP2 |
| ENSG00000172171 | TEFM | ENSG00000175832 | ETV4 |
| ENSG00000135913 | USP37 | ENSG00000185359 | HGS |
| ENSG00000135624 | CCT7 | ENSG00000120705 | ETF1 |
| ENSG00000100804 | PSMB5 | ENSG00000108384 | RAD51C |
| ENSG00000175792 | RUVBL1 | ENSG00000036257 | CUL3 |
| ENSG00000183431 | SF3A3 | ENSG00000152382 | TADA1 |
| ENSG00000108773 | KAT2A | ENSG00000114742 | WDR48 |
| ENSG00000100949 | RABGGTA | ENSG00000214026 | MRPL23 |
| ENSG00000151503 | NCAPD3 | ENSG00000105671 | DDX49 |
| ENSG00000111880 | RNGTT | ENSG00000104731 | KLHDC4 |
| ENSG00000168883 | USP39 | ENSG00000010256 | UQCRC1 |
| ENSG00000151461 | UPF2 | ENSG00000154743 | TSEN2 |
| ENSG00000105486 | LIG1 | ENSG00000178896 | EXOSC4 |
| ENSG00000111300 | NAA25 | ENSG00000168393 | DTYMK |
| ENSG00000144559 | TAMM41 | ENSG00000035928 | RFC1 |
| ENSG00000137574 | TGS1 | ENSG00000048707 | VPS13D |
| ENSG00000172273 | HINFP | ENSG00000154832 | CXXC1 |
| ENSG00000133112 | TPT1 | ENSG00000130985 | UBA1 |
| ENSG00000167986 | DDB1 | ENSG00000065150 | IPO5 |
| ENSG00000125319 | C17orf53 | ENSG00000161800 | RACGAP1 |
| ENSG00000113161 | HMGCR | ENSG00000142534 | RPS11 |
| ENSG00000100941 | PNN | ENSG00000136003 | ISCU |
| ENSG00000139697 | SBNO1 | ENSG00000065000 | AP3D1 |
| ENSG00000135336 | ORC3 | ENSG00000100401 | RANGAP1 |
| ENSG00000101115 | SALL4 | ENSG00000196230 | TUBB |
| ENSG00000100902 | PSMA6 | ENSG00000181555 | SETD2 |
| ENSG00000141141 | DDX52 | ENSG00000055950 | MRPL43 |
| ENSG00000254093 | PINX1 | ENSG00000188389 | PDCD1 |
| ENSG00000184445 | KNTC1 | ENSG00000165684 | SNAPC4 |
| ENSG00000089053 | ANAPC5 | ENSG00000147533 | GOLGA7 |
| ENSG00000111602 | TIMELESS | ENSG00000064313 | TAF2 |
| ENSG00000145592 | RPL37 | ENSG00000137154 | RPS6 |
| ENSG00000106615 | RHEB | ENSG00000104886 | PLEKHJ1 |
| ENSG00000180817 | PPA1 | ENSG00000122882 | ECD |
| ENSG00000110172 | CHORDC1 | ENSG00000184967 | NOC4L |
| ENSG00000137876 | RSL24D1 | ENSG00000088325 | TPX2 |
| ENSG00000104408 | EIF3E | ENSG00000183520 | UTP11L |
| ENSG00000143436 | MRPL9 | ENSG00000179051 | RCC2 |
| ENSG00000108883 | EFTUD2 | ENSG00000157510 | AFAP1L1 |
| ENSG00000140740 | UQCRC2 | ENSG00000066379 | ZNRD1 |
| ENSG00000211456 | SACM1L | ENSG00000172115 | CYCS |
| ENSG00000131051 | RBM39 | ENSG00000086827 | ZW10 |
| ENSG00000136758 | YME1L1 | ENSG00000109534 | GAR1 |
| ENSG00000112578 | BYSL | ENSG00000175387 | SMAD2 |
| ENSG00000163781 | TOPBP1 | ENSG00000115947 | ORC4 |
| ENSG00000106628 | POLD2 | ENSG00000010072 | SPRTN |
| ENSG00000132952 | USPL1 | ENSG00000185163 | DDX51 |
| ENSG00000168538 | TRAPPC11 | ENSG00000177370 | TIMM22 |
| ENSG00000168488 | ATXN2L | ENSG00000076924 | XAB2 |
| ENSG00000022277 | RTFDC1 | ENSG00000124562 | SNRPC |
| ENSG00000179988 | PSTK | ENSG00000127586 | CHTF18 |
| ENSG00000092199 | HNRNPC | ENSG00000066117 | SMARCD1 |
| ENSG00000156831 | NSMCE2 | ENSG00000177494 | ZBED2 |
| ENSG00000125691 | RPL23 | ENSG00000133401 | PDZD2 |
| ENSG00000083520 | DIS3 | ENSG00000127554 | GFER |
| ENSG00000115761 | NOL10 | ENSG00000117697 | NSL1 |
| ENSG00000173894 | CBX2 | ENSG00000184659 | FOXD4L4 |
| ENSG00000243147 | MRPL33 | ENSG00000204828 | FOXD4L2 |
| ENSG00000139618 | BRCA2 | ENSG00000110200 | ANAPC15 |
| ENSG00000109519 | GRPEL1 | ENSG00000169291 | SHE |
| ENSG00000203760 | CENPW | ENSG00000132313 | MRPL35 |
| ENSG00000166851 | PLK1 | ENSG00000115816 | CEBPZ |
| ENSG00000121579 | NAA50 | ENSG00000243667 | WDR92 |
| ENSG00000163608 | C3orf17 | ENSG00000107959 | PITRM1 |
| ENSG00000005075 | POLR2J | ENSG00000103035 | PSMD7 |
| ENSG00000148606 | POLR3A | ENSG00000163946 | FAM208A |
| ENSG00000160949 | TONSL | ENSG00000178057 | NDUFAF3 |
| ENSG00000128159 | TUBGCP6 | ENSG00000170540 | ARL6IP1 |
| ENSG00000125449 | ARMC7 | ENSG00000091009 | RBM27 |
| ENSG00000122406 | RPL5 | ENSG00000205609 | EIF3CL |
| ENSG00000126226 | PCID2 | ENSG00000165526 | RPUSD4 |
| ENSG00000159377 | PSMB4 | ENSG00000120314 | WDR55 |
| ENSG00000167967 | E4F1 | ENSG00000013275 | PSMC4 |
| ENSG00000141076 | CIRH1A | ENSG00000131931 | THAP1 |
| ENSG00000069248 | NUP133 | ENSG00000155660 | PDIA4 |
| ENSG00000242372 | EIF6 | ENSG00000162607 | USP1 |
| ENSG00000087269 | NOP14 | ENSG00000109606 | DHX15 |
| ENSG00000163468 | CCT3 | ENSG00000261949 | LOC100507003 |
| ENSG00000140326 | CDAN1 | ENSG00000130589 | HELZ2 |
| ENSG00000146834 | MEPCE | ENSG00000145734 | BDP1 |
| ENSG00000143222 | UFC1 | ENSG00000103194 | USP10 |
| ENSG00000110871 | COQ5 | ENSG00000076201 | PTPN23 |
| ENSG00000119285 | HEATR1 | ENSG00000140854 | KATNB1 |
| ENSG00000145386 | CCNA2 | ENSG00000164053 | ATRIP |
| ENSG00000164109 | MAD2L1 | ENSG00000167088 | SNRPD1 |
| ENSG00000185347 | C14orf80 | ENSG00000154781 | CCDC174 |
| ENSG00000134748 | PRPF38A | ENSG00000115446 | UNC50 |
| ENSG00000070061 | IKBKAP | ENSG00000177700 | POLR2L |
| ENSG00000099995 | SF3A1 | ENSG00000162063 | CCNF |
| ENSG00000100029 | PES1 | ENSG00000152904 | GGPS1 |
| ENSG00000130255 | RPL36 | ENSG00000151657 | KIN |
| ENSG00000085231 | AK6 | ENSG00000182810 | DDX28 |
| ENSG00000187145 | MRPS21 | ENSG00000006744 | ELAC2 |
| ENSG00000062650 | WAPAL | ENSG00000116898 | MRPS15 |
| ENSG00000122484 | RPAP2 | ENSG00000255072 | PIGY |
| ENSG00000090861 | AARS | ENSG00000130332 | LSM7 |
| ENSG00000161888 | SPC24 | ENSG00000051180 | RAD51 |
| ENSG00000087087 | SRRT | ENSG00000178171 | AMER3 |
| ENSG00000134910 | STT3A | ENSG00000254901 | MEF2BNB |
| ENSG00000161526 | SAP30BP | ENSG00000149925 | ALDOA |
| ENSG00000068654 | POLR1A | ENSG00000100604 | CHGA |
| ENSG00000140983 | RHOT2 | ENSG00000172602 | RND1 |
| ENSG00000184708 | EIF4ENIF1 | ENSG00000138592 | USP8 |
| ENSG00000100479 | POLE2 | ENSG00000172613 | RAD9A |
| ENSG00000134440 | NARS | ENSG00000132196 | HSD17B7 |
| ENSG00000014164 | ZC3H3 | ENSG00000151849 | CENPJ |
| ENSG00000113812 | ACTR8 | ENSG00000105221 | AKT2 |
| ENSG00000145331 | TRMT10A | ENSG00000185504 | C17orf70 |
| ENSG00000110104 | CCDC86 | ENSG00000025796 | SEC63 |
| ENSG00000164163 | ABCE1 | ENSG00000168438 | CDC40 |
| ENSG00000167863 | ATP5H | ENSG00000163918 | RFC4 |
| ENSG00000176946 | THAP4 | ENSG00000152147 | GEMIN6 |
| ENSG00000169251 | NMD3 | ENSG00000166887 | VPS39 |
| ENSG00000166226 | CCT2 | ENSG00000018625 | ATP1A2 |
| ENSG00000131747 | TOP2A | ENSG00000163346 | PBXIP1 |
| ENSG00000267673 | FDX1L | ENSG00000135966 | TGFBRAP1 |
| ENSG00000108559 | NUP88 | ENSG00000099901 | RANBP1 |
| ENSG00000104957 | CCDC130 | ENSG00000010327 | STAB1 |
| ENSG00000167522 | ANKRD11 | ENSG00000163344 | PMVK |
| ENSG00000130706 | ADRM1 | ENSG00000102921 | N4BP1 |
| ENSG00000048162 | NOP16 | ENSG00000177150 | FAM210A |
| ENSG00000159210 | SNF8 | ENSG00000158042 | MRPL17 |
| ENSG00000113360 | DROSHA | ENSG00000124659 | TBCC |
| ENSG00000108296 | CWC25 | ENSG00000113593 | PPWD1 |
| ENSG00000161395 | PGAP3 | ENSG00000188306 | LRRIQ4 |
| ENSG00000089195 | TRMT6 | ENSG00000074966 | TXK |
| ENSG00000185838 | GNB1L | ENSG00000228049 | POLR2J2 |
| ENSG00000101146 | RAE1 | ENSG00000133226 | SRRM1 |
| ENSG00000092853 | CLSPN | ENSG00000121577 | POPDC2 |
| ENSG00000107949 | BCCIP | ENSG00000130876 | SLC7A10 |
| ENSG00000159079 | C21orf59 | ENSG00000130810 | PPAN |
| ENSG00000137947 | GTF2B | ENSG00000243207 | PPAN-P2RY11 |
| ENSG00000160948 | VPS28 | ENSG00000081248 | CACNA1S |
| ENSG00000065427 | KARS | ENSG00000153201 | RANBP2 |
| ENSG00000102978 | POLR2C | ENSG00000126698 | DNAJC8 |
| ENSG00000182154 | MRPL41 | ENSG00000103018 | CYB5B |
| ENSG00000139168 | ZCRB1 | ENSG00000130816 | DNMT1 |
| ENSG00000175110 | MRPS22 | ENSG00000102103 | PQBP1 |
| ENSG00000177084 | POLE | ENSG00000120253 | NUP43 |
| ENSG00000197681 | TBC1D3 | ENSG00000164327 | RICTOR |
| ENSG00000053501 | USE1 | ENSG00000139719 | VPS33A |
| ENSG00000121879 | PIK3CA | ENSG00000168566 | SNRNP48 |
| ENSG00000108278 | ZNHIT3 | ENSG00000063244 | U2AF2 |
| ENSG00000161547 | SRSF2 | ENSG00000108423 | TUBD1 |
| ENSG00000129083 | COPB1 | ENSG00000164880 | INTS1 |
| ENSG00000012048 | BRCA1 | ENSG00000148297 | MED22 |
| ENSG00000171314 | PGAM1 | ENSG00000185825 | BCAP31 |
| ENSG00000112159 | MDN1 | ENSG00000084623 | EIF3I |
| ENSG00000174243 | DDX23 | ENSG00000066422 | ZBTB11 |
| ENSG00000096401 | CDC5L | ENSG00000119041 | GTF3C3 |
| ENSG00000128513 | POT1 | ENSG00000083093 | PALB2 |
| ENSG00000071859 | FAM50A | ENSG00000120699 | EXOSC8 |
| ENSG00000100084 | HIRA | ENSG00000166135 | HIF1AN |
| ENSG00000100813 | ACIN1 | ENSG00000188976 | NOC2L |
| ENSG00000005100 | DHX33 | ENSG00000102974 | CTCF |
| ENSG00000101158 | NELFCD | ENSG00000148229 | POLE3 |
| ENSG00000115946 | PNO1 | ENSG00000167118 | URM1 |
| ENSG00000188647 | PTAR1 | ENSG00000176386 | CDC26 |
| ENSG00000146007 | ZMAT2 | ENSG00000110063 | DCPS |
| ENSG00000241837 | ATP5O | ENSG00000089737 | DDX24 |
| ENSG00000113643 | RARS | ENSG00000119383 | PPP2R4 |
| ENSG00000162521 | RBBP4 | ENSG00000143319 | ISG20L2 |
| ENSG00000116830 | TTF2 | ENSG00000141552 | ANAPC11 |
| ENSG00000187555 | USP7 | ENSG00000155506 | LARP1 |
| ENSG00000137216 | TMEM63B | ENSG00000144867 | SRPRB |
| ENSG00000161904 | LEMD2 | ENSG00000093000 | NUP50 |
| ENSG00000241945 | PWP2 | ENSG00000107937 | GTPBP4 |
| ENSG00000134982 | APC | ENSG00000083635 | NUFIP1 |
| ENSG00000156983 | BRPF1 | ENSG00000174527 | MYO1H |
| ENSG00000164346 | NSA2 | ENSG00000124641 | MED20 |
| ENSG00000223496 | EXOSC6 | ENSG00000240694 | PNMA2 |
| ENSG00000113569 | NUP155 | ENSG00000122012 | SV2C |
| ENSG00000080986 | NDC80 | ENSG00000017260 | ATP2C1 |
| ENSG00000143374 | TARS2 | ENSG00000179965 | ZNF771 |
| ENSG00000104835 | SARS2 | ENSG00000126216 | TUBGCP3 |
| ENSG00000152253 | SPC25 | ENSG00000126814 | TRMT5 |
| ENSG00000088356 | PDRG1 | ENSG00000101945 | SUV39H1 |
| ENSG00000044574 | HSPA5 | ENSG00000182185 | RAD51B |
| ENSG00000116874 | WARS2 | ENSG00000163681 | SLMAP |
| ENSG00000204531 | POU5F1 | ENSG00000179295 | PTPN11 |
| ENSG00000004779 | NDUFAB1 | ENSG00000004487 | KDM1A |
| ENSG00000161981 | SNRNP25 | ENSG00000136100 | VPS36 |
| ENSG00000126457 | PRMT1 | ENSG00000168066 | SF1 |
| ENSG00000142507 | PSMB6 | ENSG00000197181 | PIWIL2 |
| ENSG00000164808 | SPIDR | ENSG00000128908 | INO80 |
| ENSG00000234972 | TBC1D3C | ENSG00000102144 | PGK1 |
| ENSG00000144554 | FANCD2 | ENSG00000007923 | DNAJC11 |
| ENSG00000147383 | NSDHL | ENSG00000143514 | TP53BP2 |
| ENSG00000165732 | DDX21 | ENSG00000076650 | GPATCH1 |
| ENSG00000155975 | VPS37A | ENSG00000130749 | ZC3H4 |
| ENSG00000002822 | MAD1L1 | ENSG00000062582 | MRPS24 |
| ENSG00000179271 | GADD45GIP1 | ENSG00000087085 | ACHE |
| ENSG00000101452 | DHX35 | ENSG00000197976 | AKAP17A |
| ENSG00000074071 | MRPS34 | ENSG00000100028 | SNRPD3 |
| ENSG00000169045 | HNRNPH1 | ENSG00000128731 | HERC2 |
| ENSG00000087510 | TFAP2C | ENSG00000134014 | ELP3 |
| ENSG00000105819 | PMPCB | ENSG00000181163 | NPM1 |
| ENSG00000204351 | SKIV2L | ENSG00000148444 | COMMD3 |
| ENSG00000160783 | PMF1 | ENSG00000095319 | NUP188 |
| ENSG00000152234 | ATP5A1 | ENSG00000169564 | PCBP1 |
| ENSG00000127463 | EMC1 | ENSG00000182208 | MOB2 |
| ENSG00000124228 | DDX27 | ENSG00000055070 | SZRD1 |
| ENSG00000100319 | ZMAT5 | ENSG00000182473 | EXOC7 |
| ENSG00000065183 | WDR3 | ENSG00000136930 | PSMB7 |
| ENSG00000058272 | PPP1R12A | ENSG00000107863 | ARHGAP21 |
| ENSG00000136628 | EPRS | ENSG00000197223 | C1D |
| ENSG00000163017 | ACTG2 | ENSG00000184270 | HIST2H2AB |
| ENSG00000104884 | ERCC2 | ENSG00000161036 | LRWD1 |
| ENSG00000166483 | WEE1 | ENSG00000144736 | SHQ1 |
| ENSG00000135837 | CEP350 | ENSG00000137100 | DCTN3 |
| ENSG00000104897 | SF3A2 | ENSG00000131149 | GSE1 |
| ENSG00000140598 | EFTUD1 | ENSG00000214753 | HNRNPUL2 |
| ENSG00000143774 | GUK1 | ENSG00000111358 | GTF2H3 |
| ENSG00000085721 | RRN3 | ENSG00000147677 | EIF3H |
| ENSG00000172053 | QARS | ENSG00000125676 | THOC2 |
| ENSG00000165934 | CPSF2 | ENSG00000149554 | CHEK1 |
| ENSG00000052802 | MSMO1 | ENSG00000176476 | CCDC101 |
| ENSG00000135476 | ESPL1 | ENSG00000147596 | PRDM14 |
| ENSG00000174177 | CTU2 | ENSG00000092094 | OSGEP |
| ENSG00000120438 | TCP1 | ENSG00000155393 | HEATR3 |
| ENSG00000170892 | TSEN34 | ENSG00000083845 | RPS5 |
| ENSG00000204574 | ABCF1 | ENSG00000148296 | SURF6 |
| ENSG00000175376 | EIF1AD | ENSG00000162613 | FUBP1 |
| ENSG00000146263 | MMS22L | ENSG00000182220 | ATP6AP2 |
| ENSG00000121022 | COPS5 | ENSG00000115163 | CENPA |
| ENSG00000168090 | COPS6 | ENSG00000176225 | RTTN |
| ENSG00000167491 | GATAD2A | ENSG00000176208 | ATAD5 |
| ENSG00000084072 | PPIE | ENSG00000254827 | SLC22A18AS |
| ENSG00000115268 | RPS15 | ENSG00000128708 | HAT1 |
| ENSG00000163938 | GNL3 | ENSG00000106400 | ZNHIT1 |
| ENSG00000151665 | PIGF | ENSG00000123219 | CENPK |
| ENSG00000148843 | PDCD11 | ENSG00000264424 | MYH4 |
| ENSG00000141736 | ERBB2 | ENSG00000066468 | FGFR2 |
| ENSG00000103168 | TAF1C | ENSG00000095059 | DHPS |
| ENSG00000105401 | CDC37 | ENSG00000110921 | MVK |
| ENSG00000163933 | RFT1 | ENSG00000141556 | TBCD |
| ENSG00000122085 | MTERFD2 | ENSG00000196305 | IARS |
| ENSG00000164032 | H2AFZ | ENSG00000131055 | COX4I2 |
| ENSG00000140943 | MBTPS1 | ENSG00000153789 | FAM92B |
| ENSG00000198952 | SMG5 | ENSG00000088930 | XRN2 |
| ENSG00000169021 | UQCRFS1 | ENSG00000145220 | LYAR |
| ENSG00000013810 | TACC3 | ENSG00000172809 | RPL38 |
| ENSG00000105258 | POLR2I | ENSG00000108788 | MLX |
| ENSG00000167978 | SRRM2 | ENSG00000197170 | PSMD12 |
| ENSG00000095564 | BTAF1 | ENSG00000225899 | FRG2B |
| ENSG00000138095 | LRPPRC | ENSG00000174886 | NDUFA11 |
| ENSG00000063978 | RNF4 | ENSG00000172058 | SERF1A |
| ENSG00000162368 | CMPK1 | ENSG00000205572 | SERF1B |
| ENSG00000140829 | DHX38 | ENSG00000242485 | MRPL20 |
| ENSG00000158169 | FANCC | ENSG00000089225 | TBX5 |
| ENSG00000161960 | EIF4A1 | ENSG00000149428 | HYOU1 |
| ENSG00000181222 | POLR2A | ENSG00000166595 | FAM96B |
| ENSG00000165916 | PSMC3 | ENSG00000131462 | TUBG1 |
| ENSG00000198060 | MARCH5 | ENSG00000185990 | F8A3 |
| ENSG00000149923 | PPP4C | ENSG00000197932 | F8A1 |
| ENSG00000111667 | USP5 | ENSG00000198444 | F8A2 |
| ENSG00000198755 | RPL10A | ENSG00000031823 | RANBP3 |
| ENSG00000141499 | WRAP53 | ENSG00000100353 | EIF3D |
| ENSG00000093009 | CDC45 | ENSG00000163605 | PPP4R2 |
| ENSG00000105732 | ZNF574 | ENSG00000164162 | ANAPC10 |
| ENSG00000104064 | GABPB1 | ENSG00000132153 | DHX30 |
| ENSG00000108294 | PSMB3 | ENSG00000154723 | ATP5J |
| ENSG00000130856 | ZNF236 | ENSG00000182256 | GABRG3 |
| ENSG00000133980 | VRTN | ENSG00000119487 | MAPKAP1 |
| ENSG00000149308 | NPAT | ENSG00000132394 | EEFSEC |
| ENSG00000120071 | KANSL1 | ENSG00000122952 | ZWINT |
| ENSG00000129084 | PSMA1 | ENSG00000131042 | LILRB2 |
| ENSG00000117877 | CD3EAP | ENSG00000222004 | C7orf71 |
| ENSG00000127616 | SMARCA4 | ENSG00000168802 | CHTF8 |
| ENSG00000163882 | POLR2H | ENSG00000069849 | ATP1B3 |
| ENSG00000183718 | TRIM52 | ENSG00000074582 | BCS1L |
| ENSG00000106803 | SEC61B | ENSG00000103126 | AXIN1 |
| ENSG00000114942 | EEF1B2 | ENSG00000187144 | SPATA21 |
| ENSG00000067704 | IARS2 | ENSG00000221914 | PPP2R2A |
| ENSG00000114686 | MRPL3 | ENSG00000163386 | NBPF10 |
| ENSG00000172315 | TP53RK | ENSG00000134987 | WDR36 |
| ENSG00000173120 | KDM2A | ENSG00000132300 | PTCD3 |
| ENSG00000138442 | WDR12 | ENSG00000156931 | VPS8 |
| ENSG00000145982 | FARS2 | ENSG00000165632 | TAF3 |
| ENSG00000117481 | NSUN4 | ENSG00000044115 | CTNNA1 |
| ENSG00000142676 | RPL11 | ENSG00000035403 | VCL |
| ENSG00000164615 | CAMLG | ENSG00000088256 | GNA11 |
| ENSG00000138073 | PREB | ENSG00000164334 | FAM170A |
| ENSG00000136888 | ATP6V1G1 | ENSG00000166225 | FRS2 |
| ENSG00000221829 | FANCG | ENSG00000241186 | TDGF1 |
| ENSG00000198887 | SMC5 | ENSG00000196374 | HIST1H2BM |
| ENSG00000102900 | NUP93 | ENSG00000117614 | SYF2 |
| ENSG00000108344 | PSMD3 | ENSG00000154222 | CC2D1B |
| ENSG00000023191 | RNH1 | ENSG00000101367 | MAPRE1 |
| ENSG00000143621 | ILF2 | ENSG00000188186 | LAMTOR4 |
| ENSG00000112855 | HARS2 | ENSG00000166924 | NYAP1 |
| ENSG00000110536 | PTPMT1 | ENSG00000079805 | DNM2 |
| ENSG00000165629 | ATP5C1 | ENSG00000011260 | UTP18 |
| ENSG00000166847 | DCTN5 | ENSG00000089685 | BIRC5 |
| ENSG00000104852 | SNRNP70 | ENSG00000123908 | AGO2 |
| ENSG00000203814 | HIST2H2BF | ENSG00000057935 | MTA3 |
| ENSG00000009413 | REV3L | ENSG00000100811 | YY1 |
| ENSG00000130772 | MED18 | ENSG00000064102 | ASUN |
| ENSG00000079313 | REXO1 | ENSG00000006025 | OSBPL7 |
| ENSG00000012061 | ERCC1 | ENSG00000107372 | ZFAND5 |
| ENSG00000111642 | CHD4 | ENSG00000172922 | RNASEH2C |
| ENSG00000100462 | PRMT5 | ENSG00000075089 | ACTR6 |
| ENSG00000174100 | MRPL45 | ENSG00000165119 | HNRNPK |
| ENSG00000101421 | CHMP4B | ENSG00000182518 | FAM104B |
| ENSG00000144028 | SNRNP200 | ENSG00000041802 | LSG1 |
| ENSG00000108592 | FTSJ3 | ENSG00000206557 | TRIM71 |
| ENSG00000110048 | OSBP | ENSG00000124140 | SLC12A5 |
| ENSG00000147403 | RPL10 | ENSG00000063046 | EIF4B |
| ENSG00000198783 | ZNF830 | ENSG00000126581 | BECN1 |
| ENSG00000179409 | GEMIN4 | ENSG00000171530 | TBCA |
| ENSG00000147604 | RPL7 | ENSG00000206127 | GOLGA8O |
| ENSG00000136824 | SMC2 | ENSG00000167842 | MIS12 |
| ENSG00000104889 | RNASEH2A | ENSG00000033011 | ALG1 |
| ENSG00000146282 | RARS2 | ENSG00000146670 | CDCA5 |
| ENSG00000068784 | SRBD1 | ENSG00000198856 | OSTC |
| ENSG00000137822 | TUBGCP4 | ENSG00000111605 | CPSF6 |
| ENSG00000059691 | PET112 | ENSG00000087365 | SF3B2 |
| ENSG00000066827 | ZFAT | ENSG00000135845 | PIGC |
| ENSG00000148308 | GTF3C5 | ENSG00000100220 | RTCB |
| ENSG00000170185 | USP38 | ENSG00000131876 | SNRPA1 |
| ENSG00000160201 | U2AF1 | ENSG00000115392 | FANCL |
| ENSG00000141258 | SGSM2 | ENSG00000078618 | NRD1 |
| ENSG00000172660 | TAF15 | ENSG00000025770 | NCAPH2 |
| ENSG00000145833 | DDX46 | ENSG00000117682 | DHDDS |
| ENSG00000104980 | TIMM44 | ENSG00000198844 | ARHGEF15 |
| ENSG00000097046 | CDC7 | ENSG00000132603 | NIP7 |
| ENSG00000131368 | MRPS25 | ENSG00000162377 | SELRC1 |
| ENSG00000204209 | DAXX | ENSG00000137411 | VARS2 |
| ENSG00000129696 | TTI2 | ENSG00000064886 | CHI3L2 |
| ENSG00000108848 | LUC7L3 | ENSG00000137806 | NDUFAF1 |
| ENSG00000013573 | DDX11 | ENSG00000133030 | MPRIP |
| ENSG00000105248 | CCDC94 | ENSG00000136935 | GOLGA1 |
| ENSG00000183598 | HIST2H3D | ENSG00000243927 | MRPS6 |
| ENSG00000224226 | TBC1D3B | ENSG00000046647 | GEMIN8 |
| ENSG00000090470 | PDCD7 | ENSG00000133124 | IRS4 |
| ENSG00000031698 | SARS | ENSG00000255346 | NOX5 |
| ENSG00000108270 | AATF | ENSG00000103275 | UBE2I |
| ENSG00000159111 | MRPL10 | ENSG00000165502 | RPL36AL |
| ENSG00000149806 | FAU | ENSG00000100056 | DGCR14 |
| ENSG00000188739 | RBM34 | ENSG00000167972 | ABCA3 |
| ENSG00000152684 | PELO | ENSG00000053372 | MRTO4 |
| ENSG00000174374 | WBSCR16 | ENSG00000169813 | HNRNPF |
| ENSG00000107036 | KIAA1432 | ENSG00000198258 | UBL5 |
| ENSG00000204619 | PPP1R11 | ENSG00000103245 | NARFL |
| ENSG00000091651 | ORC6 | ENSG00000183513 | COA5 |
| ENSG00000134480 | CCNH | ENSG00000174547 | MRPL11 |
| ENSG00000164151 | KIAA0947 | ENSG00000173457 | PPP1R14B |
| ENSG00000164611 | PTTG1 | ENSG00000088038 | CNOT3 |
| ENSG00000111445 | RFC5 | ENSG00000115539 | PDCL3 |
| ENSG00000127481 | UBR4 | ENSG00000118181 | RPS25 |
| ENSG00000159352 | PSMD4 | ENSG00000160075 | SSU72 |
| ENSG00000137814 | HAUS2 | ENSG00000257949 | TEN1 |
| ENSG00000105220 | GPI | ENSG00000168028 | RPSA |
| ENSG00000140521 | POLG | ENSG00000213066 | FGFR1OP |
| ENSG00000075856 | SART3 | ENSG00000143228 | NUF2 |
| ENSG00000143742 | SRP9 | ENSG00000137413 | TAF8 |
| ENSG00000163029 | SMC6 | ENSG00000124207 | CSE1L |
| ENSG00000162227 | TAF6L | ENSG00000080815 | PSEN1 |
| ENSG00000100129 | EIF3L | ENSG00000132773 | TOE1 |
| ENSG00000170348 | TMED10 | ENSG00000129460 | NGDN |
| ENSG00000182217 | HIST2H4B | ENSG00000188613 | NANOS1 |
| ENSG00000183941 | HIST2H4A | ENSG00000163636 | PSMD6 |
| ENSG00000116221 | MRPL37 | ENSG00000146232 | NFKBIE |
| ENSG00000196235 | SUPT5H | ENSG00000135902 | CHRND |
| ENSG00000161920 | MED11 | ENSG00000143641 | GALNT2 |
| ENSG00000134690 | CDCA8 | ENSG00000073969 | NSF |
| ENSG00000131153 | GINS2 | ENSG00000041982 | TNC |
| ENSG00000138018 | EPT1 | ENSG00000108256 | NUFIP2 |
| ENSG00000173141 | MRP63 | ENSG00000198911 | SREBF2 |
| ENSG00000154727 | GABPA | ENSG00000141385 | AFG3L2 |
| ENSG00000120800 | UTP20 | ENSG00000176108 | CHMP6 |
| ENSG00000114767 | RRP9 | ENSG00000257365 | FNTB |
| ENSG00000174231 | PRPF8 | ENSG00000186487 | MYT1L |
| ENSG00000137547 | MRPL15 | ENSG00000127423 | AUNIP |
| ENSG00000146576 | C7orf26 | ENSG00000112110 | MRPL18 |
| ENSG00000065268 | WDR18 | ENSG00000114650 | SCAP |
| ENSG00000147162 | OGT | ENSG00000178104 | PDE4DIP |
| ENSG00000198917 | C9orf114 | ENSG00000105656 | ELL |
| ENSG00000180822 | PSMG4 | ENSG00000186393 | KRT26 |
| ENSG00000125977 | EIF2S2 | ENSG00000124541 | RRP36 |
| ENSG00000173418 | NAA20 | ENSG00000182108 | DEXI |
| ENSG00000155561 | NUP205 | ENSG00000139133 | ALG10 |
| ENSG00000173545 | ZNF622 | ENSG00000082068 | WDR70 |
| ENSG00000127993 | RBM48 | ENSG00000151388 | ADAMTS12 |
| ENSG00000197102 | DYNC1H1 | ENSG00000172172 | MRPL13 |
| ENSG00000119392 | GLE1 | ENSG00000184979 | USP18 |
| ENSG00000174444 | RPL4 | ENSG00000239857 | GET4 |
| ENSG00000149716 | ORAOV1 | ENSG00000069345 | DNAJA2 |
| ENSG00000155876 | RRAGA | ENSG00000073050 | XRCC1 |
| ENSG00000198841 | KTI12 | ENSG00000070985 | TRPM5 |
| ENSG00000056097 | ZFR | ENSG00000158715 | SLC45A3 |
| ENSG00000227057 | WDR46 | ENSG00000172062 | SMN1 |
| ENSG00000167670 | CHAF1A | ENSG00000205571 | SMN2 |
| ENSG00000127191 | TRAF2 | ENSG00000113141 | IK |
| ENSG00000072506 | HSD17B10 | ENSG00000186105 | LRRC70 |
| ENSG00000215021 | PHB2 | ENSG00000157895 | C12orf43 |
| ENSG00000175467 | SART1 | ENSG00000166441 | RPL27A |
| ENSG00000121073 | SLC35B1 | ENSG00000106346 | USP42 |
| ENSG00000079459 | FDFT1 | ENSG00000185379 | RAD51D |
| ENSG00000143493 | INTS7 | ENSG00000116667 | C1orf21 |
| ENSG00000141543 | EIF4A3 | ENSG00000176444 | CLK2 |
| ENSG00000174197 | MGA | ENSG00000105472 | CLEC11A |
| ENSG00000131269 | ABCB7 | ENSG00000065613 | SLK |
| ENSG00000089009 | RPL6 | ENSG00000005156 | LIG3 |
| ENSG00000197780 | TAF13 | ENSG00000125459 | MSTO1 |
| ENSG00000036549 | ZZZ3 | ENSG00000139146 | FAM60A |
| ENSG00000066135 | KDM4A | ENSG00000060069 | CTDP1 |
| ENSG00000176473 | WDR25 | ENSG00000130935 | NOL11 |
| ENSG00000124614 | RPS10 | ENSG00000115677 | HDLBP |
| ENSG00000107581 | EIF3A | ENSG00000105254 | TBCB |
| ENSG00000084463 | WBP11 | ENSG00000075539 | FRYL |
| ENSG00000137656 | BUD13 | ENSG00000196747 | HIST1H2AI |
| ENSG00000183751 | TBL3 | ENSG00000181513 | ACBD4 |
| ENSG00000119537 | KDSR | ENSG00000153107 | ANAPC1 |
| ENSG00000204220 | PFDN6 | ENSG00000160211 | G6PD |
| ENSG00000170291 | ELP5 | ENSG00000111481 | COPZ1 |
| ENSG00000198563 | DDX39B | ENSG00000070761 | C16orf80 |
| ENSG00000077549 | CAPZB | ENSG00000168924 | LETM1 |
| ENSG00000255529 | POLR2M | ENSG00000105058 | FAM32A |
| ENSG00000100034 | PPM1F | ENSG00000204569 | PPP1R10 |
| ENSG00000196367 | TRRAP | ENSG00000153914 | SREK1 |
| ENSG00000167258 | CDK12 | ENSG00000161509 | GRIN2C |
| ENSG00000039123 | SKIV2L2 | ENSG00000162702 | ZNF281 |
| ENSG00000076043 | REXO2 | ENSG00000004939 | SLC4A1 |
| ENSG00000213676 | ATF6B | ENSG00000139620 | KANSL2 |
| ENSG00000058453 | CROCC | ENSG00000025293 | PHF20 |
| ENSG00000153575 | TUBGCP5 | ENSG00000158545 | ZC3H18 |
| ENSG00000110700 | RPS13 | ENSG00000142546 | NOSIP |
| ENSG00000101181 | MTG2 | ENSG00000143398 | PIP5K1A |
| ENSG00000071539 | TRIP13 | ENSG00000197958 | RPL12 |
| ENSG00000075702 | WDR62 | ENSG00000067225 | PKM |
| ENSG00000171453 | POLR1C | ENSG00000172534 | HCFC1 |
| ENSG00000090989 | EXOC1 | ENSG00000155438 | MKI67IP |
| ENSG00000037897 | METTL1 | ENSG00000166582 | CENPV |
| ENSG00000095139 | ARCN1 | ENSG00000145912 | NHP2 |
| ENSG00000078142 | PIK3C3 | ENSG00000180992 | MRPL14 |
| ENSG00000141030 | COPS3 | ENSG00000118705 | RPN2 |
| ENSG00000126249 | PDCD2L | ENSG00000163161 | ERCC3 |
| ENSG00000117408 | IPO13 | ENSG00000136819 | C9orf78 |
| ENSG00000130725 | UBE2M | ENSG00000124787 | RPP40 |
| ENSG00000175054 | ATR | ENSG00000179104 | TMTC2 |
| ENSG00000149016 | TUT1 | ENSG00000140694 | PARN |
| ENSG00000165060 | FXN | ENSG00000143751 | SDE2 |
| ENSG00000117597 | DIEXF | ENSG00000136997 | MYC |
| ENSG00000185085 | INTS5 | ENSG00000147274 | RBMX |
| ENSG00000113595 | TRIM23 | ENSG00000084693 | AGBL5 |
| ENSG00000040633 | PHF23 | ENSG00000165271 | NOL6 |
| ENSG00000178952 | TUFM | ENSG00000221838 | AP4M1 |
| ENSG00000120539 | MASTL | ENSG00000171444 | MCC |
| ENSG00000103549 | RNF40 | ENSG00000101882 | NKAP |
| ENSG00000119723 | COQ6 | ENSG00000186847 | KRT14 |
| ENSG00000171311 | EXOSC1 | ENSG00000014824 | SLC30A9 |
| ENSG00000106245 | BUD31 | ENSG00000166685 | COG1 |
| ENSG00000118046 | STK11 | ENSG00000108349 | CASC3 |
| ENSG00000125484 | GTF3C4 | ENSG00000175216 | CKAP5 |
| ENSG00000089094 | KDM2B | ENSG00000259494 | MRPL46 |
| ENSG00000121621 | KIF18A | ENSG00000028310 | BRD9 |
| ENSG00000129911 | KLF16 | ENSG00000136450 | SRSF1 |
| ENSG00000102302 | FGD1 | ENSG00000204859 | ZBTB48 |
| ENSG00000135679 | MDM2 | ENSG00000165209 | STRBP |
| ENSG00000185115 | NDNL2 | ENSG00000163466 | ARPC2 |
| ENSG00000140553 | UNC45A | ENSG00000125485 | DDX31 |
| ENSG00000129562 | DAD1 | ENSG00000070778 | PTPN21 |
| ENSG00000100138 | NHP2L1 | ENSG00000126001 | CEP250 |
| ENSG00000111641 | NOP2 | ENSG00000169249 | ZRSR2 |
| ENSG00000173660 | UQCRH | ENSG00000111011 | RSRC2 |
| ENSG00000198677 | TTC37 | ENSG00000139496 | NUPL1 |
| ENSG00000135503 | ACVR1B | ENSG00000131746 | TNS4 |
| ENSG00000180998 | GPR137C | ENSG00000061936 | SFSWAP |
| ENSG00000153187 | HNRNPU | ENSG00000196584 | XRCC2 |
| ENSG00000106459 | NRF1 | ENSG00000168286 | THAP11 |
| ENSG00000156261 | CCT8 | ENSG00000119787 | ATL2 |
| ENSG00000118363 | SPCS2 | ENSG00000182446 | NPLOC4 |
| ENSG00000164134 | NAA15 | ENSG00000071462 | WBSCR22 |
| ENSG00000060642 | PIGV | ENSG00000213397 | HAUS7 |
| ENSG00000090889 | KIF4A | ENSG00000178028 | DMAP1 |
| ENSG00000101361 | NOP56 | ENSG00000067596 | DHX8 |
| ENSG00000167792 | NDUFV1 | ENSG00000198015 | MRPL42 |
| ENSG00000184162 | NR2C2AP | ENSG00000133706 | LARS |
| ENSG00000128524 | ATP6V1F | ENSG00000149635 | OCSTAMP |
| ENSG00000100387 | RBX1 | ENSG00000117505 | DR1 |
| ENSG00000110906 | KCTD10 | ENSG00000155868 | MED7 |
| ENSG00000147457 | CHMP7 | ENSG00000129197 | RPAIN |
| ENSG00000124570 | SERPINB6 | ENSG00000065978 | YBX1 |
| ENSG00000186468 | RPS23 | ENSG00000260238 | PMF1-BGLAP |
| ENSG00000136122 | BORA | ENSG00000178988 | MRFAP1L1 |
| ENSG00000047249 | ATP6V1H | ENSG00000168005 | C11orf84 |
| ENSG00000127804 | METTL16 | ENSG00000162408 | NOL9 |
| ENSG00000104412 | EMC2 | ENSG00000140350 | ANP32A |
| ENSG00000173726 | TOMM20 | ENSG00000261796 | ISY1-RAB43 |
| ENSG00000138777 | PPA2 | ENSG00000174405 | LIG4 |
| ENSG00000170043 | TRAPPC1 | ENSG00000197414 | GOLGA6L1 |
| ENSG00000124486 | USP9X | ENSG00000116062 | MSH6 |
| ENSG00000105705 | SUGP1 | ENSG00000116906 | GNPAT |
| ENSG00000223501 | VPS52 | ENSG00000134597 | RBMX2 |
| ENSG00000107815 | C10orf2 | ENSG00000071994 | PDCD2 |
| ENSG00000100109 | TFIP11 | ENSG00000112742 | TTK |
| ENSG00000136271 | DDX56 | ENSG00000106636 | YKT6 |
| ENSG00000146830 | GIGYF1 | ENSG00000101773 | RBBP8 |
| ENSG00000198382 | UVRAG | ENSG00000103061 | SLC7A6OS |
| ENSG00000160285 | LSS | ENSG00000140259 | MFAP1 |
| ENSG00000137770 | CTDSPL2 | ENSG00000197077 | KIAA1671 |
| ENSG00000116670 | MAD2L2 | ENSG00000204435 | CSNK2B |
| ENSG00000165280 | VCP | ENSG00000055130 | CUL1 |
| ENSG00000183963 | SMTN | ENSG00000100209 | HSCB |
| ENSG00000164961 | KIAA0196 | ENSG00000113048 | MRPS27 |
| ENSG00000157216 | SSBP3 | ENSG00000189403 | HMGB1 |
| ENSG00000129932 | DOHH | ENSG00000173011 | TADA2B |
| ENSG00000167721 | TSR1 | ENSG00000169836 | TACR3 |
| ENSG00000188352 | FOCAD | ENSG00000133816 | MICAL2 |
| ENSG00000104853 | CLPTM1 | ENSG00000141452 | C18orf8 |
| ENSG00000185883 | ATP6V0C | ENSG00000006715 | VPS41 |
| ENSG00000100519 | PSMC6 | ENSG00000136518 | ACTL6A |
| ENSG00000110107 | PRPF19 | ENSG00000100297 | MCM5 |
| ENSG00000184203 | PPP1R2 | ENSG00000165898 | ISCA2 |
| ENSG00000148824 | MTG1 | ENSG00000156384 | SFR1 |
| ENSG00000113810 | SMC4 | ENSG00000145414 | NAF1 |
| ENSG00000121152 | NCAPH | ENSG00000101972 | STAG2 |
| ENSG00000241127 | YAE1D1 | ENSG00000112658 | SRF |
| ENSG00000139197 | PEX5 | ENSG00000162736 | NCSTN |
| ENSG00000101464 | PIGU | ENSG00000103266 | STUB1 |
| ENSG00000132676 | DAP3 | ENSG00000008018 | PSMB1 |
| ENSG00000135972 | MRPS9 | ENSG00000149506 | ZP1 |
| ENSG00000089157 | RPLP0 | ENSG00000111530 | CAND1 |
| ENSG00000138035 | PNPT1 | ENSG00000027001 | MIPEP |
| ENSG00000171824 | EXOSC10 | ENSG00000152266 | PTH |
| ENSG00000153179 | RASSF3 | ENSG00000154174 | TOMM70A |
| ENSG00000110713 | NUP98 | ENSG00000164045 | CDC25A |
| ENSG00000100865 | CINP | ENSG00000164758 | MED30 |
| ENSG00000136045 | PWP1 | ENSG00000160401 | C9orf117 |
| ENSG00000167526 | RPL13 | ENSG00000155959 | VBP1 |
| ENSG00000088766 | CRLS1 | ENSG00000105409 | ATP1A3 |
| ENSG00000103510 | KAT8 | ENSG00000175106 | TVP23C |
| ENSG00000143368 | SF3B4 | ENSG00000185950 | IRS2 |
| ENSG00000156697 | UTP14A | ENSG00000149256 | TENM4 |
| ENSG00000176248 | ANAPC2 | ENSG00000116957 | TBCE |
| ENSG00000188786 | MTF1 | ENSG00000154719 | MRPL39 |
| ENSG00000175756 | AURKAIP1 | ENSG00000105364 | MRPL4 |
| ENSG00000140395 | WDR61 | ENSG00000198218 | QRICH1 |
| ENSG00000113368 | LMNB1 | ENSG00000013503 | POLR3B |
| ENSG00000060339 | CCAR1 | ENSG00000126756 | UXT |
| ENSG00000162385 | MAGOH | ENSG00000184988 | TMEM106A |
| ENSG00000105372 | RPS19 | ENSG00000186432 | KPNA4 |
| ENSG00000083312 | TNPO1 | ENSG00000156304 | SCAF4 |
| ENSG00000100142 | POLR2F | ENSG00000090565 | RAB11FIP3 |
| ENSG00000204560 | DHX16 | ENSG00000163508 | EOMES |
| ENSG00000197771 | MCMBP | ENSG00000147003 | TMEM27 |
| ENSG00000099817 | POLR2E | ENSG00000198730 | CTR9 |
| ENSG00000161980 | POLR3K | ENSG00000105321 | CCDC9 |
| ENSG00000117133 | RPF1 | ENSG00000120333 | MRPS14 |
| ENSG00000125901 | MRPS26 | ENSG00000121680 | PEX16 |
| ENSG00000168827 | GFM1 | ENSG00000088205 | DDX18 |
| ENSG00000161513 | FDXR | ENSG00000132432 | SEC61G |
| ENSG00000137818 | RPLP1 | ENSG00000186329 | TMEM212 |
| ENSG00000150990 | DHX37 | ENSG00000094804 | CDC6 |
| ENSG00000061794 | MRPS35 | ENSG00000169084 | DHRSX |
| ENSG00000143155 | TIPRL | ENSG00000107618 | RBP3 |
| ENSG00000253626 | EIF5AL1 | ENSG00000146426 | TIAM2 |
| ENSG00000231500 | RPS18 | ENSG00000198925 | ATG9A |
| ENSG00000188076 | SCGB1C1 | ENSG00000168242 | HIST1H2BI |
| ENSG00000174442 | ZWILCH | ENSG00000254772 | EEF1G |
| ENSG00000242028 | HYPK | ENSG00000090971 | NAT14 |
| ENSG00000124217 | MOCS3 | ENSG00000144381 | HSPD1 |
| ENSG00000134186 | PRPF38B | ENSG00000127774 | EMC6 |
| ENSG00000105849 | TWISTNB | ENSG00000126259 | KIRREL2 |
| ENSG00000137337 | MDC1 | ENSG00000111364 | DDX55 |
| ENSG00000132207 | SLX1A | ENSG00000100749 | VRK1 |
| ENSG00000181625 | SLX1B | ENSG00000159063 | ALG8 |
| ENSG00000110717 | NDUFS8 | ENSG00000163795 | ZNF513 |
| ENSG00000132341 | RAN | ENSG00000068394 | GPKOW |
| ENSG00000014123 | UFL1 | ENSG00000112659 | CUL9 |
| ENSG00000101191 | DIDO1 | ENSG00000187257 | RSBN1L |
| ENSG00000125952 | MAX | ENSG00000172167 | MTBP |
| ENSG00000163714 | U2SURP | ENSG00000176177 | ENTHD1 |
| ENSG00000253710 | ALG11 | ENSG00000166783 | KIAA0430 |
| ENSG00000104356 | POP1 | ENSG00000165006 | UBAP1 |
| ENSG00000130826 | DKC1 | ENSG00000188958 | UTS2B |
| ENSG00000198780 | FAM169A | ENSG00000136247 | ZDHHC4 |
| ENSG00000116688 | MFN2 | ENSG00000196363 | WDR5 |
| ENSG00000166166 | TRMT61A | ENSG00000116661 | FBXO2 |
| ENSG00000214517 | PPME1 | ENSG00000113013 | HSPA9 |
| ENSG00000077235 | GTF3C1 | ENSG00000090061 | CCNK |
| ENSG00000152240 | HAUS1 | ENSG00000051596 | THOC3 |
| ENSG00000063177 | RPL18 | ENSG00000140534 | TICRR |
| ENSG00000087157 | PGS1 | ENSG00000100216 | TOMM22 |
| ENSG00000100567 | PSMA3 | ENSG00000104613 | INTS10 |
| ENSG00000169371 | SNUPN | ENSG00000183474 | GTF2H2C |
| ENSG00000197651 | CCER1 | ENSG00000159128 | IFNGR2 |
| ENSG00000198900 | TOP1 | ENSG00000243725 | TTC4 |
| ENSG00000213551 | DNAJC9 | ENSG00000102898 | NUTF2 |
| ENSG00000152464 | RPP38 | ENSG00000170515 | PA2G4 |
| ENSG00000131467 | PSME3 | ENSG00000117036 | ETV3 |
| ENSG00000223510 | CDRT15 | ENSG00000196262 | PPIA |
| ENSG00000115053 | NCL | ENSG00000153037 | SRP19 |
| ENSG00000163041 | H3F3A | ENSG00000135801 | TAF5L |
| ENSG00000154813 | DPH3 | ENSG00000119414 | PPP6C |
| ENSG00000181873 | IBA57 | ENSG00000141013 | GAS8 |
| ENSG00000185591 | SP1 | ENSG00000113845 | TIMMDC1 |
| ENSG00000115355 | CCDC88A | ENSG00000175826 | CTDNEP1 |
| ENSG00000139350 | NEDD1 | ENSG00000117543 | DPH5 |
| ENSG00000108518 | PFN1 | ENSG00000204779 | FOXD4L5 |
| ENSG00000108264 | TADA2A | ENSG00000112249 | ASCC3 |
| ENSG00000134809 | TIMM10 | ENSG00000152256 | PDK1 |
| ENSG00000124383 | MPHOSPH10 | ENSG00000169217 | CD2BP2 |
| ENSG00000126067 | PSMB2 | ENSG00000166246 | C16orf71 |
| ENSG00000060688 | SNRNP40 | ENSG00000184164 | CRELD2 |
| ENSG00000042429 | MED17 | ENSG00000107960 | OBFC1 |
| ENSG00000196655 | TRAPPC4 | ENSG00000102384 | CENPI |
| ENSG00000107185 | RGP1 | ENSG00000079785 | DDX1 |
| ENSG00000124608 | AARS2 | ENSG00000133858 | ZFC3H1 |
| ENSG00000092098 | RNF31 | ENSG00000184110 | EIF3C |
| ENSG00000143569 | UBAP2L | ENSG00000146700 | SRCRB4D |
| ENSG00000233822 | HIST1H2BN | ENSG00000163380 | LMOD3 |
| ENSG00000171848 | RRM2 | ENSG00000116273 | PHF13 |
| ENSG00000183161 | FANCF | ENSG00000178229 | ZNF543 |
| ENSG00000166197 | NOLC1 | ENSG00000109475 | RPL34 |
| ENSG00000064703 | DDX20 | ENSG00000156469 | MTERFD1 |
| ENSG00000176102 | CSTF3 | ENSG00000155827 | RNF20 |
| ENSG00000106028 | SSBP1 | ENSG00000213741 | RPS29 |
| ENSG00000143315 | PIGM | ENSG00000165792 | METTL17 |
| ENSG00000136152 | COG3 | ENSG00000110844 | PRPF40B |
| ENSG00000134697 | GNL2 | ENSG00000100842 | EFS |
| ENSG00000159217 | IGF2BP1 | ENSG00000087495 | PHACTR3 |
| ENSG00000080608 | KIAA0020 | ENSG00000126261 | UBA2 |
| ENSG00000267368 | UPK3BL | ENSG00000136718 | IMP4 |
| ENSG00000130119 | GNL3L | ENSG00000091640 | SPAG7 |
| ENSG00000178950 | GAK | ENSG00000184886 | PIGW |
| ENSG00000205659 | LIN52 | ENSG00000184313 | MROH7 |
| ENSG00000123297 | TSFM | ENSG00000163481 | RNF25 |
| ENSG00000241370 | RPP21 | ENSG00000137054 | POLR1E |
| ENSG00000129351 | ILF3 | ENSG00000213085 | CCDC19 |
| ENSG00000174446 | SNAPC5 | ENSG00000171858 | RPS21 |
| ENSG00000132382 | MYBBP1A | ENSG00000130822 | PNCK |
| ENSG00000100664 | EIF5 | ENSG00000145216 | FIP1L1 |
| ENSG00000131469 | RPL27 | ENSG00000147130 | ZMYM3 |
| ENSG00000185128 | TBC1D3F | ENSG00000008086 | CDKL5 |
| ENSG00000111231 | GPN3 | ENSG00000165282 | PIGO |
| ENSG00000182774 | RPS17L | ENSG00000038358 | EDC4 |
| ENSG00000184779 | RPS17 | ENSG00000134684 | YARS |
| ENSG00000186871 | ERCC6L | ENSG00000153832 | FBXO36 |
| ENSG00000204568 | MRPS18B | ENSG00000140006 | WDR89 |
| ENSG00000108312 | UBTF | ENSG00000104643 | MTMR9 |
| ENSG00000167965 | MLST8 | ENSG00000151779 | NBAS |
| ENSG00000115241 | PPM1G | ENSG00000077348 | EXOSC5 |
| ENSG00000171103 | TRMT61B | ENSG00000131043 | AAR2 |
| ENSG00000116586 | LAMTOR2 | ENSG00000160193 | WDR4 |
| ENSG00000105793 | GTPBP10 | ENSG00000140691 | ARMC5 |
| ENSG00000100348 | TXN2 | ENSG00000141959 | PFKL |
| ENSG00000172757 | CFL1 | ENSG00000112053 | SLC26A8 |
| ENSG00000163634 | THOC7 | ENSG00000197111 | PCBP2 |
| ENSG00000008324 | SS18L2 | ENSG00000145191 | EIF2B5 |
| ENSG00000152404 | CWF19L2 | ENSG00000140988 | RPS2 |
| ENSG00000020129 | NCDN | ENSG00000181472 | ZBTB2 |

The gene symbols used herein (including in Tables 2 and 3) are based on those found in the Human Gene Naming Committee (HGNC) which is searchable on the world-wide web at genenames.org. Ensembl IDs are provided for each gene symbol and are searchable world-wide web at ensembl.org.

The genes provided in Tables 2 and 3 are non-limiting examples of essential genes. Although additional essential genes will be apparent to the skilled artisan based on the knowledge in the art, the suitability of a particular gene for use according to the present disclosure may be determined, e.g., by analysis of potential off-target sites elsewhere in the genome. In some aspects, only essential genes with one or more gRNA target sites that are unique in the human genome are selected for the method and system described herein. In some aspects, only essential genes with one or more gRNA target sites that are found in only one other locus in the human genome are selected for use in the method and system described herein. In some aspects, only essential genes with one or more gRNA target sites found in only two other loci in the human genome are selected for the method and system described herein.

In some aspects, an editing construct and/or a reporter construct may be provided or introduced into cells as double-stranded nucleic acid molecules or single-stranded nucleic acid molecules. In some aspects, an editing construct and/or a reporter construct may be provided to cells as a plasmid, vector (e.g., viral vector), or oligonucleotide (e.g., an ssODN).

In some aspects, if a portion of the coding sequence of the gene encoding the essential protein is not integrated into the genome of the cells by HDR in the correct position or orientation, the cells do not survive and/or proliferate. However, if the portion of the coding sequence of the gene encoding the essential protein is integrated into the genome of the cells by HDR in the correct position or orientation, the cells survive and/or proliferate and are therefore selected for based upon growth and/or proliferation. Successful editing of the essential gene at the same time as editing a genomic location of interest allows for the essential gene to function as a surrogate reporter for successful editing at the genomic location of interest. Accordingly, cells that survive and/or proliferate are indicative of cells that are edited.

In some aspects herein, a break in accordance with the method herein is a double-strand break. In some aspects, a break is a single-strand break. In some aspects, a break in accordance with the method herein is induced by contacting the cells with a nuclease, i.e., the nuclease cleaves the genomic location to cause a break. The term “nuclease” as used herein refers to any protein that catalyzes the cleavage of phosphodiester bonds. In some embodiments the nuclease is a DNA nuclease. In some aspects, the nuclease is a “nickase,” which causes a single-strand break when it cleaves double-stranded DNA, e.g., genomic DNA in a cell. In some aspects, the nuclease causes a double-strand break when it cleaves double-stranded DNA, e.g., genomic DNA in a cell. In some aspects, the nuclease is a prime editor comprising a nickase domain fused to a reverse transcriptase domain. In some aspects, the nuclease is an RNA-guided prime editor and the gRNA comprises the donor template. In some aspects, a dual-nickase system is used, which causes a double-strand break via two single-strand breaks on opposing strands of a double-stranded DNA, e.g., genomic DNA of the cell. In some aspects, the nuclease binds a specific target site within the double-stranded DNA that overlaps with or is adjacent to the location of the resulting break. In some aspects, the nuclease causes a double-strand break that contains overhangs ranging from 0 (blunt ends) to 22 nucleotides in both 3′ and 5′ orientations.

In some aspects, the nuclease is a CRISPR/Cas nuclease. In some aspects, a CRISPR/Cas nuclease may include, but is not limited to, naturally-occurring Class 2 CRISPR nucleases such as Cas9, and Cpf1 (Cas12a), as well as other Cas12 nucleases and nucleases derived or obtained therefrom. In functional terms, CRISPR/Cas nucleases are defined as those nucleases that: (a) interact with (e.g., complex with) a gRNA; and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif,” or “PAM.” CRISPR/Cas nucleases may be defined, in broad terms, by their PAM specificity and cleavage activity, even though variations may exist between individual CRISPR/Cas nucleases that share the same PAM specificity or cleavage activity. Skilled artisans will appreciate that some aspects of the present disclosure relate to systems and methods that may be implemented using any suitable CRISPR/Cas nuclease having a certain PAM specificity and/or cleavage activity. For this reason, unless otherwise specified, the term CRISPR/Cas nuclease should be understood as a generic term, and not limited to any particular type (e.g., Cas9 vs. Cpf1), species (e.g., Streptococcus pyogenes vs. Staphylococcus aureus) or variation (e.g., full-length vs. truncated or split; naturally-occurring PAM specificity vs. engineered PAM specificity, etc.) of CRISPR/Cas nuclease.

The PAM sequence takes its name from its sequential relationship to the “protospacer” sequence that is complementary to gRNA targeting domains (or “spacers”). Together with protospacer sequences, PAM sequences define target regions or sequences for specific CRISPR/Cas nuclease and gRNA combinations.

Various CRISPR/Cas nucleases may require different sequential relationships between PAMS and protospacers. In general, Cas9s recognize PAM sequences that are 3′ of the protospacer. Cpf1 (Cas12a), on the other hand, generally recognizes PAM sequences that are 5′ of the protospacer.

In addition to recognizing specific sequential orientations of PAMs and protospacers, CRISPR/Cas nucleases may also recognize specific PAM sequences. S. aureus Cas9, for instance, recognizes a PAM sequence of NNGRRT or NNGRRV, wherein the N residues are immediately 3′ of the region recognized by the gRNA targeting domain. S. pyogenes Cas9 recognizes NGG PAM sequences. Francisella tularensis Cpf1 recognizes a TTN PAM sequence. PAM sequences have been identified for a variety of CRISPR/Cas nucleases, and a strategy for identifying novel PAM sequences has been described by Shmakov et al. ((2015) Molecular Cell 60:385-397). It should also be noted that engineered CRISPR/Cas nucleases may have PAM specificities that differ from the PAM specificities of reference molecules (for instance, in the case of an engineered CRISPR/Cas nuclease, the reference molecule may be the naturally occurring variant from which the CRISPR/Cas nuclease is derived, or the naturally occurring variant having the greatest amino acid sequence homology to the engineered CRISPR/Cas nuclease).

When using a CRISPR/Cas nuclease in the method and/or system herein, some aspects further provide for contacting the cells with a guide nucleic acid that binds to the genomic location of interest and a guide nucleic acid that binds to genomic location within the coding sequence of the gene encoding the essential protein. Guide RNAs (gRNAs) of the present disclosure may be unimolecular (comprising a single RNA molecule, and referred to alternatively as chimeric), or modular (comprising more than one, and typically two, separate RNA molecules, such as a crRNA and a tracrRNA, which are usually associated with one another, for instance by duplexing). gRNAs and their component parts are described throughout the literature, for instance in Briner et al. (2014) Molecular Cell 56(2):333-339 and in WO 2016/073990 A1.

As used herein, guide RNAs, whether unimolecular or modular, include a “targeting domain” that is fully or partially complementary to a target domain within a genomic location (e.g., genomic location of interest or genomic location within the coding sequence of an essential gene). Targeting domains are referred to by various names in the literature, including without limitation “guide sequences” (Hsu et al. (2013) Nat. Biotechnol. 31(9):827-832), “complementarity regions” (WO 2016/073990 A1), “spacers” (Briner et al. (2014) Molecular Cell 56(2):333-339) and generically as “crRNAs” (Jiang et al. (2013) Nat. Biotechnol. 31(3):233-239). Irrespective of the names they are given, targeting domains are typically 10-30 nucleotides in length, and in certain aspects are 16-24 nucleotides in length (for instance, 16, 17, 18, 19, 20, 21, 22, 23 or 24 nucleotides in length), and are at or near the 5′ terminus of in the case of a Cas9 gRNA, and at or near the 3′ terminus in the case of a Cpf1 gRNA.

Those of skill in the art will appreciate that although structural differences may exist between gRNAs from different prokaryotic species, or between Cpf1 and Cas9 gRNAs, the principles by which gRNAs operate are generally consistent. Because of this consistency of operation, gRNAs may be defined, in broad terms, by their targeting domain sequences, and skilled artisans will appreciate that a given targeting domain sequence may be incorporated in any suitable gRNA, including a unimolecular or chimeric gRNA, or a gRNA that includes one or more chemical modifications and/or sequential modifications (substitutions, additional nucleotides, truncations, etc.). Thus, for brevity, gRNAs may be described solely in terms of their targeting domain sequences.

More generally, skilled artisans will appreciate that some aspects of the present disclosure relate to systems, methods, and compositions that may be implemented using multiple CRISPR/Cas nucleases. For this reason, unless otherwise specified, the term gRNA should be understood to encompass any suitable gRNA that may be used with any CRISPR/Cas nuclease, and not only those gRNAs that are compatible with a particular species of Cas9 or Cpf1. By way of illustration, the term gRNA may, in certain aspects, include a gRNA for use with any CRISPR/Cas nuclease occurring in a Class 2 CRISPR system, such as a type II or type V or CRISPR system, or a CRISPR/Cas nuclease derived or adapted therefrom.

In some aspects, a method or system of the present disclosure may use more than one gRNA to target more than one essential gene and/or more than one genomic location of interest. In some aspects, two or more gRNAs may be used to create two or more double-strand breaks in the genome of a cell. In some aspects, a multiplexed editing strategy may be used that targets two or more essential genes at the same time with two or more replacement constructs and/or two or more genomic locations of interest with two or more editing constructs. In some aspects, the two or more editing constructs may comprise different exogenous nucleotide sequences of interest encoding different gene products of interest (e.g., transgenes) and thus the edited cells will express a plurality of gene products of interest from different exogenous nucleotide sequences targeted to different genomic locations of interest.

In some aspects, the method and system herein include the use of a zinc finger nuclease (ZFN), a transcription activator-like effector nuclease (TALEN), a meganuclease, or other nuclease known in the art (or a combination thereof) to induce or introduce a break at a genomic location (e.g., at a genomic location of interest and/or at a genomic location within a coding sequence of a gene encoding an essential protein). Methods for designing zinc finger nucleases (ZFNs) are well known in the art, e.g., see Urnov et al. (2010) Nature Reviews Genetics 11:636-640 and Paschon et al. (2019) Nat. Commun. 10(1):1133. Methods for designing transcription activator-like effector nucleases (TALENs) are well known in the art, e.g., see Joung & Sander (2013) Nat. Rev. Mol. Cell Biol. 14(1):49-55. Methods for designing meganucleases are also well known in the art, e.g., see Silva et al. (2011) Curr. Gene Ther. 11(1):11-27 and Redel & Prather (2016) Toxicol. Pathol. 44(3):428-433.

In some aspects, the disclosure also provides an edited cell or population of edited cells produced by the method herein, or progeny thereof. In some aspects, an edited cell produced the method described herein may be a yeast cell, an algal cell, a plant cell, an insect cell, a non-human animal cell, an isolated and purified human cell, and a mammalian tissue culture cell. In some aspects, an edited cell is an edited primary progenitor, edited ES cell or edited iPS cell.

In some aspects, an edited cell or population of edited cells herein may be used in the treatment of a disease, disorder, or condition. Accordingly, also provided herein is a method of preventing or treating a disease, disorder, or condition in a subject in need thereof by administering to the subject an edited cell or the pharmaceutical composition comprising the edited cell. In some aspects, the edited cell may be administered by a delivery route selected from the group of intravenous, intraperitoneal, intramuscular, intradermal, subcutaneous, intrathecal, intraosseous, and a combination thereof. In some aspects, the disease, disorder, or condition is a hemoglobinopathy (e.g., sickle cell disease, α-thalassemia, β-thalassemia, or δ-thalassemia), a viral infection (e.g., a hepatitis B virus infection, hepatitis C virus infection, human papilloma virus infection, human immunodeficiency virus (HIV) infection, human T-lymphotrophic virus (HTLV) infection, Epstein-Barr virus infection, herpes virus infection, cytomegalovirus infection, and any other chronic viral infection), X-linked severe combined immune deficiency, Fanconi anemia, hemophilia, neoplasia, cancer, amyotrophic lateral sclerosis (ALS), Alzheimer's disease, Parkinson's disease, cystic fibrosis, blood diseases and disorders, inflammation, immune system diseases or disorders, metabolic diseases, liver diseases and disorders, kidney diseases and disorders, muscular diseases and disorders (e.g., Becker muscular dystrophy, Duchenne muscular dystrophy, Emery-Dreifuss muscular dystrophy, facioscapulohumeral muscular dystrophy, any other muscular dystrophy, and muscular atrophy), bone or cartilage diseases and disorders, neurological and neuronal diseases and disorders, cardiovascular diseases and disorders, pulmonary diseases and disorders, or lysosomal storage disorders.

For use in the method herein, also provided is a system or kit for editing the genome of a cell comprising (a) a nuclease that introduces a break at a genomic location of interest and at a genomic location within a coding sequence of a gene encoding an essential protein required for survival and/or proliferation of the cells; (b) an editing construct comprising an exogenous nucleotide sequence of interest flanked by at least two regions of homology to the genomic location of interest; and (c) a replacement construct comprising a portion of the coding sequence of the gene encoding the essential protein flanked by at least two regions of homology to the genomic location within the coding sequence of the gene encoding the essential protein, wherein the portion of the coding sequence of the gene encoding the essential protein comprises at least one silent mutation. In some aspects, the at least two regions of homology to the genomic location of interest correspond to sequences located on either side of the break at the genomic location of interest, and the at least two regions of homology to the genomic location within the coding sequence of the gene encoding the essential protein correspond to sequences located on either side of the break at the genomic location within a coding sequence of the gene encoding the essential protein.

In some aspects, the nuclease is a CRISPR/Cas nuclease and the system further comprises (d) a guide nucleic acid that binds to the genomic location of interest and (e) a guide nucleic acid that binds to genomic location within the coding sequence of the gene encoding the essential protein.

In some aspects, the nuclease is a zinc finger nuclease (ZEN), a transcription activator-like effector nuclease (TALEN) or a meganuclease.

In some aspects, the essential protein is encoded by a housekeeping gene. In some aspects, the cells are induced pluripotent stem (iPS) cells or embryonic stem (ES) cells and the essential gene is involved in differentiation of iPS or ES cells or expansion of iPS-derived or ES-derived cells.

What is claimed is:

1. A method for producing and enriching for edited cells in a population of cells comprising
inducing in the genome of cells of a population a break
at a genomic location of interest and
at a genomic location within a coding sequence of a gene encoding an essential protein required for survival and/or proliferation of the cells;
providing to the cells an editing construct comprising an exogenous nucleotide sequence of interest flanked by at least two regions of homology to the genomic location of interest;
providing to the cells a replacement construct comprising a portion of the coding sequence of the gene encoding the essential protein flanked by at least two regions of homology to the genomic location within the coding sequence of the gene encoding the essential protein, wherein the portion of the coding sequence of the gene encoding the essential protein comprises at least one silent mutation; and
selecting cells of the population of cells that comprise the exogenous nucleotide sequence of interest and express the essential protein thereby producing and enriching for edited cells in the population of cells.

2. The method of claim 1, wherein, if the portion of the coding sequence of t the gene encoding the essential protein is not integrated into the genome of the cells by homology-directed repair (HDR) in the correct position or orientation, the cells do not survive and/or proliferate.

3. The method of claim 1, wherein the break is a double-strand break.

4. The method of claim 1, wherein the break is induced by contacting the cells with a nuclease.

5. The method of claim 4, wherein the nuclease is a CRISPR/Cas nuclease and the method further comprises contacting the cells with a guide nucleic acid that binds to the genomic location of interest and a guide nucleic acid that binds to genomic location within the coding sequence of the gene encoding the essential protein.

6. The method of claim 4, wherein the nuclease is a zinc finger nuclease (ZFN), a transcription activator-like effector nuclease (TALEN) or a meganuclease.

7. The method of claim 1, wherein the at least two regions of homology to the genomic location of interest correspond to sequences located on either side of the break at the genomic location of interest, and the at least two regions of homology to the genomic location within the coding sequence of the gene encoding the essential protein correspond to sequences located on either side of the break at the genomic location within a coding sequence of the gene encoding the essential protein.

8. The method of claim 1, wherein integration of the portion of the coding sequence of the gene encoding the essential protein into the genome of the cells by homology-directed repair (HDR) does not alter the amino acid sequence and/or function of the essential protein.

9. The method of claim 1, wherein the essential protein is encoded by a housekeeping gene.

10. The method of claim 1, wherein the cells are induced pluripotent stem (iPS) cells or embryonic stem (ES) cells and the essential protein is involved in differentiation of iPS or ES cells or expansion of iPS-derived or ES-derived cells.

11. An edited cell or population of edited cells produced by the method of claim 1, or progeny thereof.

12. The edited cell or population of cells of claim 11, wherein edited cell or population of edited cells are used in the treatment of a disease, disorder, or condition.

13. A system for editing the genome of a cell comprising
a nuclease that introduces a break at a genomic location of interest and at a genomic location within a coding sequence of a gene encoding an essential protein required for survival and/or proliferation of the cells;
an editing construct comprising an exogenous nucleotide sequence of interest flanked by at least two regions of homology to the genomic location of interest; and
a replacement construct comprising a portion of the coding sequence of the gene encoding the essential protein flanked by at least two regions of homology to the genomic location within the coding sequence of the gene encoding the essential protein, wherein the portion of the coding sequence of the gene encoding the essential protein comprises at least one silent mutation.

14. The system of claim 13, wherein the nuclease is a CRISPR/Cas nuclease and the system further comprises a guide nucleic acid that binds to the genomic location of interest and a guide nucleic acid that binds to genomic location within the coding sequence of the gene encoding the essential protein.

15. The system of claim 13, wherein the nuclease is a zinc finger nuclease (ZFN), a transcription activator-like effector nuclease (TALEN) or a meganuclease.

16. The system of claim 13, wherein the at least two regions of homology to the genomic location of interest correspond to sequences located on either side of the break at the genomic location of interest, and the at least two regions of homology to the genomic location within the coding sequence of the gene encoding the essential protein correspond to sequences located on either side of the break at the genomic location within a coding sequence of the gene encoding the essential protein.

17. The system of claim 13, wherein the essential protein is encoded by a housekeeping gene.

18. The system of claim 13, wherein the cells are induced pluripotent stem (iPS) cells or embryonic stem (ES) cells and the essential protein is involved in differentiation of iPS or ES cells or expansion of iPS-derived or ES-derived cells.
